(19) United States
(12) Patent Application Publication
(10) Pub. No.: US 2005/0282159 A1
(43) Pub. Date: Dec. 22, 2005

(54) Rpob gene of streptomyces, primer specific to streptomyces, and identification method of streptomyces having rifampin resistance or sensitivity by using the same

(76) Inventors: Bum-Joon Kim, Jeju-city (KR); Moo-Jae Cho, Jeju-city (KR); Yoon-Ho Kook, Seoul (KR); Jeong-Mi Kim, Seoul (KR); Jung-Min Park, Seoul (KR); Chang-Jin Kim, Daejeon (KR)

Correspondence Address: KNOBBE MARTENS OLSON & BEAR LLP, 2040 MAIN STREET, FOURTEENTH FLOOR, IRVINE, CA, 92614, US

(21) Appl. No.: 10/486,669

(22) PCT Filed: Jul. 11, 2002
(86) PCT No.: PCT/KR02/01318

§ 371 Date: Jun. 20, 2005

(30) Foreign Application Priority Data
Aug. 14, 2001 (KR) 2001/48983
Jun. 28, 2002 (KR) 2002/36731
Jul. 8, 2002 (KR) 2002/39464

(51) Int. Cl. C12Q 1/68; C07H 21/04; C12N 9/22

(52) U.S. Cl. 435/6; 435/199; 536/23.2

(57) ABSTRACT

The present invention relates to a polynucleotide having a 306-bp fragment of an ANA polymerase gene subunit B (rpoB) of Streptomyces, and an identifying method of Streptomyces species using the same. According to the identifying method, the Streptomyces can be detected or identified accurately, economically, and easily. In addition, the identifying method of rifampin-resistant and rifampin-sensitive Streptomyces is a molecular-biological method having advantages in efficiency in terms of cost and time, and accuracy, and which can be widely used for identifying the Streptomyces species in the future.

### BACKGROUND OF THE INVENTION

(a) Field of the Invention

The present invention relates to an rpoB gene fragment of the Streptomyces species, primers specific to the rpoB gene of the Streptomyces species, and a method of identifying the Streptomyces species, and a rifampin-resistant strain and a rifampin-sensitive strain, by using the same.

(b) Description of the Related Art

The genus Streptomyces covers various kinds of species, and has a different physiological metabolism between the same species. Thus, various biologically-active substances are developed from metabolites of Streptomyces, and Streptomyces has many potential applications for agriculture and fisheries (breeding, control of pathogens), the environmental industry (waste decomposition), the fine chemical industry (industrial chemicals), the food industry (raw materials, additives etc.), the semiconductor industry (biosensors), the medical field, etc. Recently, more and more studies have reported that natural products can be used for preventing, alleviating, or treating various diseases. One of the objectives of studies on natural products is to first obtain various biological sources. Considering the various physiologies and industrial applications, Streptomyces can be applied to various fields (Hutchinson C, Colmbo A. Genetic engineering of doxorubicin production in Streptomyces peucetius: J Ind Microbiol Biotechnol. 1999 23(1): 647-652).

Streptomyces can be classified according to numerical taxonomy based on phenotypic, physiological, morphological, or biochemical characteristics. However, because Streptomyces consists of various species, and grows slowly compared with the other microorganisms, it is difficult to classify by using biochemical or physiological taxonomy (Skerman, V. B. D., McGowan, V., Sneath, P. H. A. (Eds.): Approved Lists of Bacterial Names. Int. J. Syst. Bacteriol. 30:225-420 (1980)).

In addition, the general tendency is that molecular taxonomy can be used for identifying species by analyzing nucleotide sequences of chronometer molecules showing a phylogenetic relationship. Thus, the molecular taxonomy using comparative sequence analysis for 16S rDNA and other targets has disadvantages in problems of target genes, cost, and time, thereby making it difficult to identify species accurately (Ueda K, Seki T, Kudo T, Yoshida T, Kataoka M. Two distinct mechanisms cause heterogeneity of 16S rRNA. J. Bacteriol. 1999 January; 181(1):78-82). For example, a 16 rDNA target gene of the Streptomyces species must first be amplified through a PCR, the amplified product must be cloned into vectors to produce clones, and then the nucleotide sequence of the clone is analyzed, because the sequence of the amplified product cannot be used directly.

Accordingly, in addition to 16S rDNA, an alternative chronometer molecule useful for identifying Streptomyces and a simple and accurate identification method using the alternative chronometer are still required.

In the approximately 70 years since the development of streptomycin, pharmaceutical companies have made an effect to separate new strains of Streptomyces from soil to produce new biologically-active substances. Without a unique method that is different from the old isolation method, it is very difficult to isolate and separate a new strain from soil and produce a new biologically-active material. Thus, as a useful method for isolating a new strain, antibiotic-resistant strains can be selected from various Streptomyces in soil by using antibiotic selection pressure (Bormann C et al., J. Antibiot, 1989, 42(6):913-8). However, because the mechanism of Streptomyces resistance to each antibiotic is not well established, there is no screening method for Streptomyces in using the molecular biological method based on antibiotics resistance of Streptomyces.

It is known that various target genes are involved in resistance to antibiotics such as streptomycin and isoniazid, and new target genes which are now known are considered to involve a resistance mechanism to antibiotics other than streptomycin and isoniazid (Zhang et al., Trends Microbiol. 1993,1(3): 109-13. Review; Riley L W et al., Clin Infect Dis. 1993; 17 (2): 442-446). In the case of screening Streptomyces in a medium, target genes associated with antibiotic resistance are present in various regions of the selection strain, thereby make it very difficult to determine whether antibiotics cause nucleotide change or not by using the molecular biological method.

### SUMMARY OF THE INVENTION

It is one object of the present invention to provide polynucleotides that are 306-bp fragments or parts thereof of an RNA polymerase β-subunit (rpoB) of Streptomyces.

It is another object of the present invention to provide primers specific for rpoB genes of Streptomyces.

It is yet another object of the present invention to provide a method for identifying Streptomyces species by using a 306-bp rpoB fragment.

It is still another object of the present invention to provide a method for identifying a rifampin-resistant strain and a rifampin-sensitive strain by using differences in nucleotide sequences of rpoB genes of Streptomyces.

It is a further object of the present invention to provide primers for specifically amplifying a rifampin-resistant Streptomyces or a rifampin-sensitive Streptomyces.

### BRIEF DESCRIPTION OF THE DRAWINGS

FIG. 1 shows phylogenetic relationships with respect to 306-bp rpoB fragments of sequenced bacteria.

FIG. 2 shows nucleotide sequences of primers specific to Streptomyces according to the present invention, and rpoB sequences of 4 strains including S. coelicolor (GenBank No. AL160431), M. smegmatis (GenBank No. U24494), M. tuberculosis (GenBank No. L27989), and M. leprae (GenBank No. Z14314).

FIG. 3 is a photograph of electrophoresis showing 352-bp products of reference strains amplified by performing PCR reaction with the primers (SRPOF1, SRPOR1) specific to Streptomyces.

FIG. 4 shows a result of analysis of nucleotide sequence similarity and homology for 24 strains of Streptomyces.

FIGS. 5a and 5b show a phylogenetic tree based on the nucleotide sequences of the 306-bp rpoB gene fragment of 102 reference strains of Streptomyces.

FIGS. 6a to 6d show results of identifying 8 non-reference strains with the comparative sequence analysis of the rpoB 306-bp fragment, wherein FIG. 6a is for the S. olivichromogenes (KCTC9090) strain, FIG. 6b is for two S. peucetius strains (KCTC 9038, KCTC 9242), FIG. 6c is for three S. hydroscopicus strains (KCTC 9030, KCTC 9031, KCTC 9069), and FIG. 6d is for two S. albus strains (KCTC 1136, KCTC 1533).

FIG. 7 is a photograph showing a culture of a rifampin-resistant strain and a rifampin-sensitive strain.

FIG. 8 shows primers specific for the rpoB gene of Streptomyces, rifampin-resistant Streptomyces, or rifampin-sensitive Streptomyces.

FIG. 9 is an electropherogram obtained by amplifying a 306-bp rpoB gene fragment of Streptomyces with the primers specific for Streptomyces, and sequencing with an automated DNA sequencer.

FIG. 10 is a photograph showing the results of identifying a rifampin-resistant strain and a rifampin-sensitive strain by using the primers specific for a rifampin-resistant or a rifampin-sensitive Streptomyces.

### DETAILED DESCRIPTION OF THE PREFERRED EMBODIMENT

The present invention relates to a polynucleotide which is a 306-bp rpoB gene fragment or parts thereof of Streptomyces, a method for identifying Streptomyces by using the rpoB gene fragments, primers specific for rifampin-resistant Streptomyces and rifampin-sensitive Streptomyces, and a method for identifying rifampin-resistant Streptomyces and rifampin-sensitive Streptomyces by using the rpoB gene fragments and primers.

To resolve the problems of a method for identifying and detecting the Streptomyces species, the inventors provided a PCR primer set for amplifying rpoB genes of all Streptomyces, and established a database containing gene fragments of RNA polymerase (-subunits (rpoB)) as a new chronometer from 162 reference strains of Streptomyces with the primers. The rpoB gene fragments of strains of interest were amplified and then compared with the database to detect and identify the Streptomyces species. In addition, the inventors discovered that, depending on the specific nucleotide sequence in an rpoB gene fragment, Streptomyces could be divided into two groups of a rifampin-resistant strain and a rifampin-sensitive strain to be used for a method for differentiating and identifying of Streptomyces.

The present invention relates to a polynucleotide of rpoB gene fragments for detecting or identifying the Streptomyces species. More specifically, the present invention relates to a polynucleotide comprising the nucleotide sequence selected from the group consisting of SEQ ID NO: 6 to SEQ ID NO: 167, a 306-bp fragment of rpoB, or parts thereof.

It was observed that all 162 reference strains produced a 352-bp PCR product when a PCR reaction was run with the primers specific for the rpoB gene of Streptomyces. The PCR products according to the present invention were determined to be new sequences by comparing them with the GenBank database. In order to amplify the 352-bp rpoB DNA fragments of 162 Streptomyces reference strains, the strains as shown in Table 1a and 1b were selected as reference strains. The strains were provided by the Korean Collection for Type Cultures of the Korea Research Institute of Bioscience and Biotechnology, at # 52, Oun-dong, Yusong-ku, Taejon Korea.

TABLE 1a

| No. | Species | Source |
|---|---|---|
| 1 | K. azatica | KCTC 9699 |
| 2 | K. cystarginea | KCTC 9746 |
| 3 | K. griseola | KCTC 9671 |
| 4 | K. mediocidica | KCTC 9733 |
| 5 | K. phosalacinea | KCTC 9792 |
| 6 | K. setae | KCTC 9793 |
| 7 | M. echinospora | KCTC 9549 |
| 8 | S. abikoensis | KCTC 9662 |
| 9 | S. achromogenes | KCTC 1740 |
| 10 | S. acrimycini | KCTC 9679 |
| 11 | S. actuosus | KCTC 9112 |
| 12 | S. aculeolatus | KCTC 9680 |
| 13 | S. alanosinicus | KCTC 9683 |
| 14 | S. albireticuli | KCTC 9685 |
| 15 | S. albofaciens | KCTC 9747 |
| 16 | S. alboflavus | KCTC 9674 |
| 17 | S. albogriseolus | KCTC 9773 |
| 18 | S. albolongus | KCTC 9676 |
| 19 | S. alboniger | KCTC 9014 |
| 20 | S. albosporeus | KCTC 9666 |
| 21 | S. alboviridis | KCTC 9750 |
| 22 | S. albulus | KCTC 9668 |
| 23 | S. albus | KCTC 1082 |
| 24 | S. almquistil | KCTC 9751 |
| 25 | S. aminophilus | KCTC 9673 |
| 26 | S. antimycoticus | KCTC 9694 |
| 27 | S. argenteolus | KCTC 9695 |
| 28 | S. armeniacus | KCTC 9120 |
| 29 | S. avidinii | KCTC 9757 |
| 30 | S. bacillaris | KCTC 9018 |
| 31 | S. bambergiensis | KCTC 9019 |
| 32 | S. bikiniensis | KCTC 9172 |
| 33 | S. cacoi asoensis | KCTC 9700 |
| 34 | S. capillispiralis | KCTC 1719 |
| 35 | S. carpinensis | KCTC 9128 |
| 36 | S. catenulae | KCTC 9223 |
| 37 | S. celluloflavus | KCTC 9702 |
| 38 | S. chartreusis | KCTC 9704 |
| 39 | S. chattanoogensis | KCTC 1087 |
| 40 | S. chrysomallus | KCTC 9705 |
| 41 | S. cinereoruber | KCTC 9707 |
| 42 | S. cinereus | KCTC 9066 |
| 43 | S. cinnamonensis | KCTC 9708 |
| 44 | S. cirratus | KCTC 9709 |
| 45 | S. clavuligerus | KCTC 9095 |
| 46 | S. coelicolor | KCTC 9005 |
| 47 | S. coeruleorubidus | KCTC 1743 |
| 48 | S. collinus | KCTC 9713 |
| 49 | S. corchorusii | KCTC 9715 |
| 50 | S. crystallinus | KCTC 9717 |
| 51 | S. cuspidosporus | KCTC 9718 |
| 52 | S. cyaneus | KCTC 9719 |
| 53 | S. diasticus | KCTC 9142 |
| 54 | S. djakartensis | KCTC 9722 |
| 55 | S. durhamensis | KCTC 9723 |
| 56 | S. echinoruber | KCTC 9725 |
| 57 | S. ederensis | KCTC 9726 |
| 58 | S. ehimensis | KCTC 9728 |
| 59 | S. flaveolus | KCTC 9737 |
| 60 | S. flavofuscus | KCTC 9760 |
| 61 | S. fradiae | KCTC 1919 |
| 62 | S. galilaeus | KCTC 9026 |
| 63 | S. globisporus | KCTC 9027 |
| 64 | S. | KCTC 9028 |
| | griseochromogenes | |
| 65 | S. griseolus | KCTC 9780 |
| 66 | S. griseoviridis | KCTC 9080 |
| 67 | S. griseus | KCTC 9781 |
| 68 | S. hiroshimensis | KCTC 9782 |
| 69 | S. hygroscopicus | KCTC 9113 |
| 70 | S. libani libani | KCTC 9033 |
| 71 | S. limosus | KCTC 1868 |
| 72 | S. lincolnensis | KCTC 9022 |
| 73 | S. longwoodensis | KCTC 9783 |
| 74 | S. melanogenes | KCTC 9205 |
| 75 | S. minutiscleroticus | KCTC 9123 |
| 76 | S. nitrosporeus | KCTC 9761 |
| 77 | S. noboritoensis | KCTC 9060 |
| 78 | S. nodosus | KCTC 9035 |
| 79 | S. nojiriensis | KCTC 9784 |
| 80 | S. olivaceoviridis | KCTC 9132 |

TABLE 1b

| No. | Species | Source |
|---|---|---|
| 81 | S. olivochromogenes | KCTC 9064 |
| 82 | S. pactum | KCTC 9165 |
| 83 | S. paradoxus | KCTC 9118 |
| 84 | S. peucetius | KCTC 9199 |
| 85 | S. phaeochromogenes | KCTC 9763 |
| 86 | S. plicatus | KCTC 9040 |
| 87 | S. pulveraceus | KCTC 9766 |
| 88 | S. rameus | KCTC 9767 |
| 89 | S. rimosus | KCTC 1077 |
| 90 | S. roseosporus | KCTC 9568 |
| 91 | S. sclerotialus | KCTC 9065 |
| 92 | S. setonii | KCTC 9144 |
| 93 | S. siovaensis | KCTC 9043 |
| 94 | S. somaliensis | KCTC 9044 |
| 95 | S. spectabilis | KCTC 9218 |
| 96 | S. subrutilus | KCTC 9045 |
| 97 | S. tubercidicus | KCTC 9109 |
| 98 | S. vinaceus | KCTC 9771 |
| 99 | S. violarus | KCTC 9788 |
| 100 | S. violascens | KCTC 9785 |
| 101 | S. virginiae | KCTC 1747 |
| 102 | S. xantophaeus | KCTC 9220 |
| 103 | S. albaduncus | KCTC 1741 |
| 104 | S. althioticus | KCTC 9752 |
| 105 | S. ambofaciens | KCCM 40182 |
| 106 | S. anulatus | KCCM 40190 |
| 107 | S. anthocyanicus | KCTC 9755 |
| 108 | S. cellulose | KCTC 9703 |
| 109 | S. chivaensis | KCTC 9786 |
| 110 | S. coelescens | KCCM 40742 |
| 111 | S. griseoflavus | KCCM 12624 |
| 112 | S. humiferus | KCTC 9116 |
| 113 | S. lividans | KCTC 1154 |
| 114 | S. murinus | KCTC 9492 |
| 115 | S. pilosus | KCCM 40480 |
| 116 | S. rubiginosus | KCTC 9042 |
| 117 | S. tendae | KCCM 40105 |
| 118 | S. umbrinus | KCCM 40316 |
| 119 | S. violaceoruber | KCTC 9787 |
| 120 | S. xanthocidicus | KCCM 40286 |
| 121 | S. yokosukanens | KCCM 40633 |
| 122 | S. amakusaensis | KCTC 9753 |
| 123 | S. aburaviensis | KCTC 9663 |
| 124 | S. albospinus | KCTC 9762 |
| 125 | S. albovinaceous | KCCM 40177 |
| 126 | S. anabdii | KCTC 9687 |
| 127 | S. antibioticus | KCTC 1137 |
| 128 | S. atroolvaceous | KCTC 9017 |
| 129 | S. aureufaciens | KCCM 40127 |
| 130 | S. azureus | KCCM 40485 |
| 131 | S. baldacii | KCCM 41326 |
| 132 | S. candidus | KCTC 9020 |
| 133 | S. caseius | KCCM 40740 |
| 134 | S. californicus | KCCM 40605 |
| 135 | S. carpinensis | KCTC 9128 |
| 136 | S. chromogenes | KCCM 40727 |
| 137 | S. cinnamoneus | KCCM 40572 |
| 138 | S. citreofluorescens | KCTC 9710 |
| 139 | S. coerulescens | KCCM 40508 |
| 140 | S. coeruleofuscus | KCCM 40506 |
| 141 | S. coralus | KCCM 40642 |
| 142 | S. cremeus | KCCM 40509 |
| 143 | S. cyaneofuscatus | KCCM 40517 |
| 144 | S. disatochromogenes | KCCM 40449 |
| 145 | S. erumpens | KCTC 9729 |
| 146 | S. erythraeus | KCCM 40477 |
| 147 | S. eurythermus | KCTC 9731 |
| 148 | S. fimbriatus | KCCM 11888 |
| 149 | S. flavotricini | KCCM 40520 |
| 150 | S. flavovirens | KCCM 40165 |
| 151 | S. fulvissimus | KCTC 9773 |
| 152 | S. fumanus | KCCM 40522 |
| 153 | S. gougeroti | KCCM 40681 |
| 154 | S. griseoruber | KCCM 40658 |
| 155 | S. griseolosporeus | KCTC 9791 |
| 156 | S. griseostramineus | KCCM 40526 |
| 157 | S. hachijoense | KCCM 32306 |
| 158 | S. halstedii | KCCM 40613 |
| 159 | S. humidus | KCCM 40647 |
| 160 | S. indigoferus | KCCM 40495 |
| 161 | S. kifunensis | KCTC 9734 |
| 162 | S. kurssanovi | KCCM 40527 |

TABLE 2

| No. | Species | Source |
|---|---|---|
| 1 | S. olivichromogenes | KCTC 9090 |
| 2 | S. peucetius | KCTC 9038 |
| 3 | S. peucetius | KCTC 9242 |
| 4 | S. hydroscopicus | KCTC 9030 |
| 5 | S. hydroscopicus | KCTC 9031 |
| 6 | S. hydroscopicus | KCTC 9069 |
| 7 | S. albus | KCTC 1136 |
| 8 | S. albus | KCTC 1533 |

For detecting and identifying the Streptomyces, the present invention provides 306-bp rpoB gene fragments encoding RNA polymerase subunit B as a new chronometer molecule, instead of 16S rDNA. The chronometer molecules must satisfy the following requirements to reflect the phylogenetic relationship.

Firstly, the target gene is essential for functions and is highly conserved in all organisms. 16S rDNA, which is essential for protein synthesis, is relatively conserved in all organisms, and genetic mutation between 16s rDNA of bacteria can be used for understanding the chronometer relationship in evolution. The target gene of the present invention, the rpoB gene which is essential for gene transcription, can satisfy the requirement.

Secondly, genetic variation of the target gene must only be caused by a temporal factor. That is, the nucleotide sequence does not change by lateral transfer based on selection pressure between species. The target gene of the present invention, the rpoB gene, is not mutated by lateral transfer based on selection pressure between species.

Thirdly, the target gene must have interspecies variation and intraspecies conservation, which suitably reflects a phylogenetic relationship. Studies report that the rpoB gene satisfied the requirements (Kim B J, et al., J Clin Microbiol, 37(6), pp. 1714-20,1999; Lee S H, et al., J Clin Microbiol. 38(7), pp. 2557-62, 2000).

The 306-bp of an rpoB gene fragment is surrounded by a highly-conserved region 5 and 6 (HCR5, HCR6) of which amino acid sequences are highly conserved in eubacteria. Thus, based on the putative nucleotide sequence of the conserved regions, it is possible to design the primers specific for Streptomyces. In addition, the 306-bp of an rpoB gene fragment has been known to link with rifampin-resistance Mycobacterium tuberculosis and E. coli (Telenti, A. P. Imboden, F. Marchesi, D. Lowrie, S. Cole, M. J. Colston, L. Matter, K. Schopfer, and T. Bodmer. 1993. Detection of rifampin-resistance mutations in Mycobacterium tuberculosis. Lancet 341:647-650).

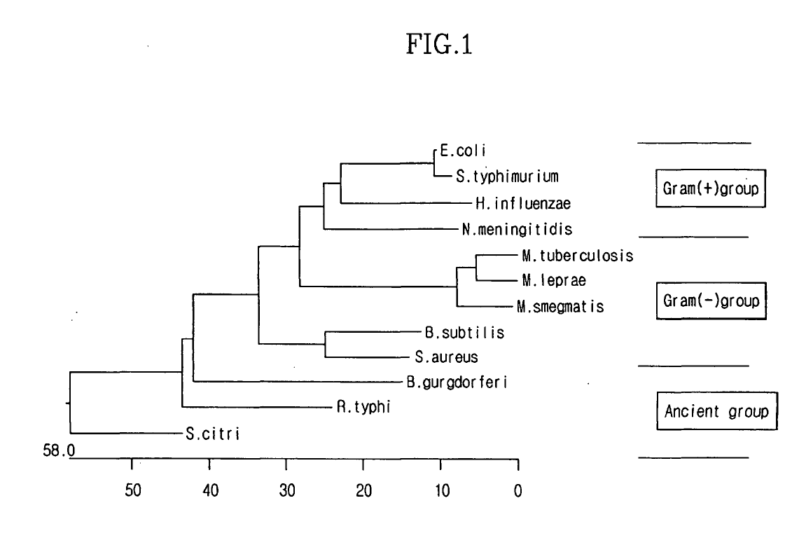

To determine whether the 306-bp of an rpoB gene fragment is preferred for an evolutionary chronometer, a phylogenetic tree was constructed based on 306-bp gene fragments derived from rpoB genes of several bacteria of which nucleotide sequences were analyzed (FIG. 1). As shown in FIG. 1, the bacteria are divided into three groups of Gram-positive, Gram-negative, and an ancient group.

Compared with 16S rDNA which has been widely used as a chronometer, the 306-bp of rpoB gene fragments provides an accurate method of detecting strains by only sequencing the 306-bp fragment, thereby providing efficiency in cost and time. The present invention also has advantages in that a clone does not need to be sequenced, the rpoB gene fragment does not contain a gap, and the nucleotides are the same size.

In addition, according to the present invention, interest bacteria can be identified by using a 306-bp rpoB gene fragment of Streptomyces, for example in comparing the sequences of a 306-bp fragment of a reference strain and an rpoB gene of an interest bacteria by applying molecular biological methods based on differences in nucleotide sequences, and through this method, Streptomyces species are detected or identified. Examples of using the method include nucleotide sequencing of rpoB, an identifying method by hybridizing using a 306-bp fragment of Streptomyces or parts thereof as a probe, or an analyzing method by fixing a probe comprising an rpoB gene of Streptomyces or parts thereof onto a microarray and contacting an amplified product for an rpoB gene thereto.

In addition, the present invention relates to a method for identifying Streptomyces species by using an rpoB gene fragment which comprises the steps of:
(1) amplifying an rpoB gene fragment of a strain of interest in a sample with primers for specifically amplifying rpoB genes of Streptomyces;
(2) analyzing a nucleotide sequence of the amplified rpoB gene fragment; and
(3) comparing the nucleotide sequence obtained in step (2) with rpoB 306-bp fragments of reference strains. Preferably, the step (3) is performed by comparing a nucleotide sequence selected from the group consisting of nucleotide sequences set forth in SEQ ID NO: 6 to SEQ ID NO: 167 with the nucleotide sequence obtained in step (2).

In step (1), the primer set contains any primer set that can specifically 15 amplify the rpoB gene of Streptomyces, and it preferably includes nucleotide sequences consisting of SEQ ID NO: 1 to SEQ ID NO:2.

The primer set specific for the rpoB fragment of Streptomyces can be designed by comparing the rpoB of Mycobacterium species, which have the closest relationship with Streptomyces, and selecting the most highly-conserved sequence as a forward primer and a backward primer. In addition, the primers can be selected so that the amplified product includes a region related with rifampin resistance of Mycobacterium tuberculosis and E. coli.

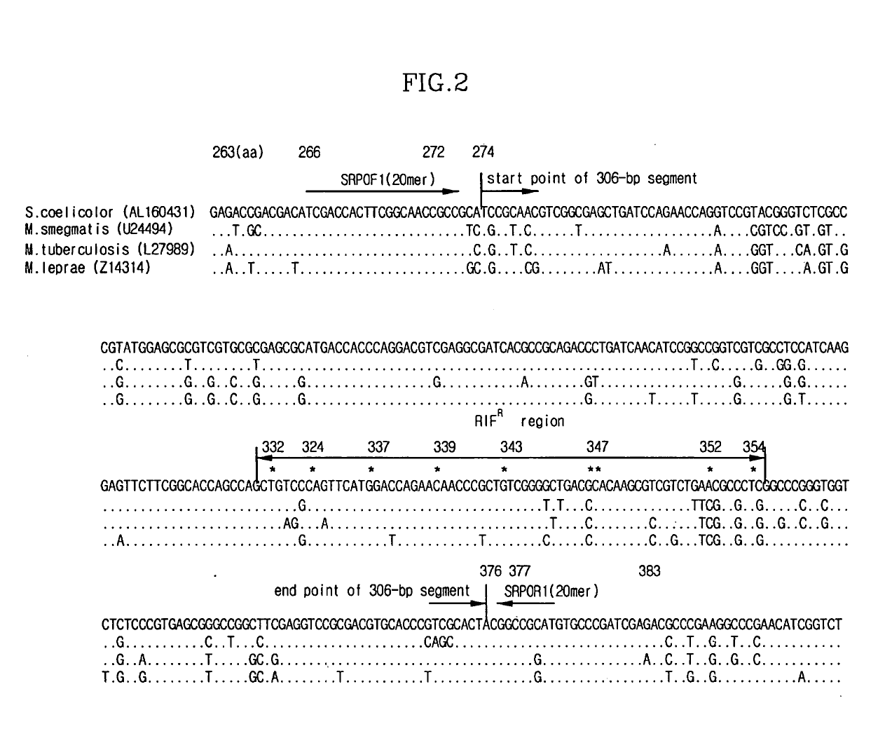

To prepare the primers for specifically amplifying rpoB genes of all Streptomyces, rpoB gene sequences of S. coelicolor (GenBank No. AL160431)), M. smegmatis (GenBank No. U24494)), M. tuberculosis (GenBank No. L27989), and M. leprae (GenBank No. Z14314), which have already been analyzed in GenBank, are comparatively analyzed. The forward primer contains 20 base (5′-TC GAC CAC TTC GGC AAC CGC-3′) located at the 2^{nd} nucleotide position of the 266 codon to the 3^{rd} nucleotide position of the 273 codon of S. coelicolor, and the forward primer is called SRPOF1 (FIG. 2).

The backward primer can be selected from 20 base nucleotide sequences having 100% nucleotide sequence homology with M. smegmatis, which belongs to the rapid growing mycobacteria group (FIG. 2). The backward primer is called SRPOR1 (5′-TC GAT CGG GCA CAT GCG GCC-3′), and it has a nucleotide sequence located at the 2^{nd} nucleotide position of the 383 codon to the 1^{st} nucleotide position of the 377 codon from 3′ to 5′ in an rpoB gene of M. smegmatis.

An rpoB gene of the strain of interest can be amplified by using primers specific for Streptomyces, and then analyzing by sequencing. PCR and nucleotide sequencing methods, which are known to an ordinary person skilled in the field can also be applied to the present invention (Kim B J, et al., J Clin Microbiol, 37(6), pp. 1714-20,1999).

The phylogenetic tree or nucleotide sequence homology can be used for identifying the strain of interest in a sample by sequencing rpoB gene fragments of the strain of interest and comparing the sequences. That is, the nucleotide sequence of the strain of interest can be introduced to the database obtained by multi-sequence alignment of the reference strain with sequence analysis software, and then the multi-sequence alignment can be performed for the sequences (for example, 162 reference sequences plus a sample DNA sequence) to complete the phylogenetic tree. When a nucleotide sequence of a strain of interest has at least 99.7% nucleotide sequence homology with that of the reference strain, it can be identified as a reference strain. This is because interspecies genetic variation between all organisms is at least 3%, and intra-species sequence homology is at least 99.7%.

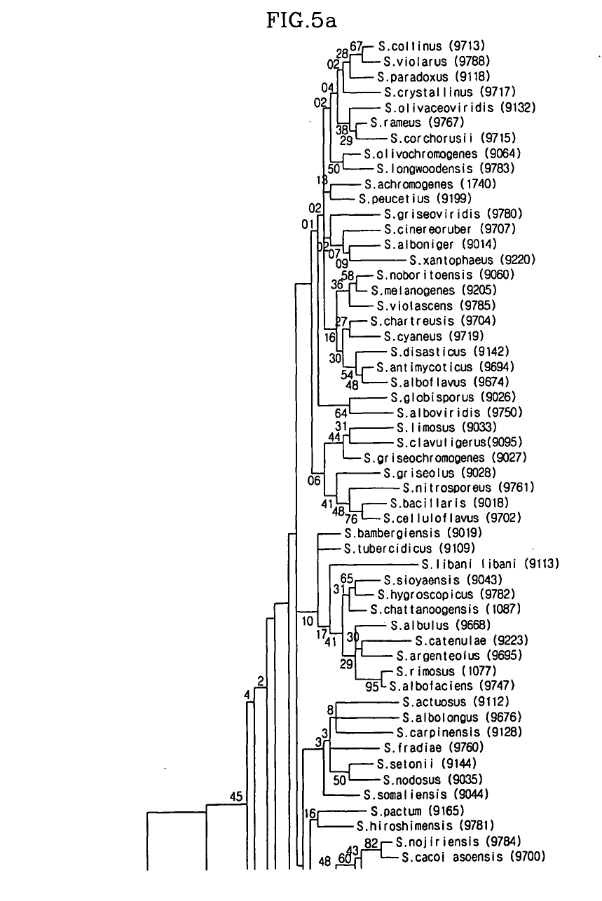

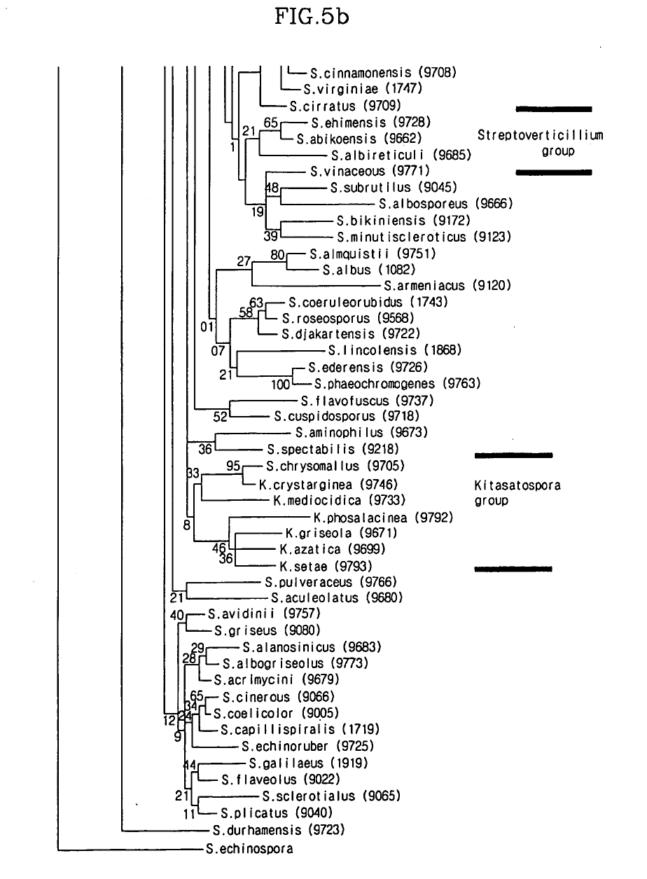

In the present invention, a Neighbor-Joining phylogenetic tree was constructed by introducing the multiply aligned nucleotide of 162 strains into Mega software. In the phylogenetic tree, it was shown that all 162 strains had different nucleotide sequences, and 162 unique branches were formed. Also, 161 Streptomyces strains were united to the exclusion of M. echinospora (FIG. 5). In a Streptomyces group of 161 strains, Kitasatospora, which is not taxonomically defined, and Streptoverticillium formed a small group within the Streptomyces group. Both groups were reported to belong to different species because cells had different physiology and biochemical characteristics, for example in meso-diaminopimelic acid content, while in molecular taxonomy by 16S rDNA, the groups are not independent genus but part of the group among Streptomyces (Zhang Z, et al., Int J Syst Bacteriol, 47(4), pp. 1048-54,1997).

When the phylogenetic tree determined by 306-bp rpoB genes was compared with classifications according to known molecular biology, similar results can be obtained. That is, 6 strains including K. azatica, K. crystarginea, K. griseola, K. mediocidica, K. phosalcinea, K. setae, and 3 strains including S. abikoensis, S. albirecticuli, S. ehimensis, belong to a subgroup in Streptomyces, but they do not generate an independent branched small group as M. echinospora (FIG. 5).

Thus, 306-bp rpoB gene fragments of the present invention are good chronometer molecules due to successfull reflection of phylogenetic relationships in Streptomyces. In considering that chronometer molecules that reflect a phylogenetic relationship well are suitable for identification purposes, 306-bp rpoB gene fragments can be successfully applied to identify Streptomyces species.

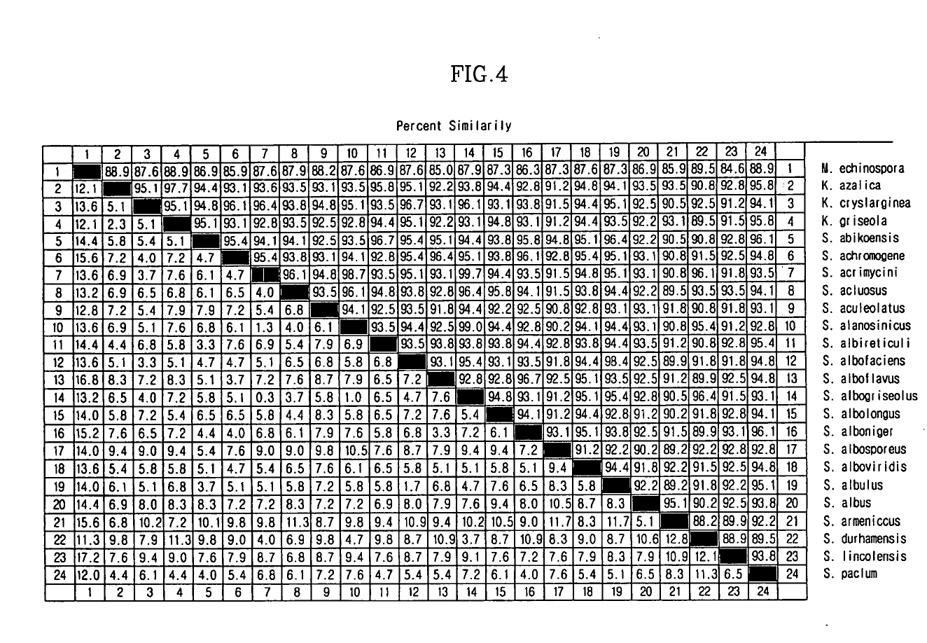

After multi-alignment of nucleotide sequences, homology between nucleotide sequences of 162 reference strains was investigated. As a result, 162 strains were found to have various amounts of homology. The homology was from 99.7% (homology between S. acrimycini and S. albogriseolus) to 84.6% (homology between M. echinospora and S. lincolensis) (FIG. 4). The homology between 161 strains of Streptomyces and M. echinospora was less than 90% (84.6-88.9%). FIG. 4 was prepared by selecting 24 strains of Streptomyces, and the strains were selected in order to contain the lowest value and the highest value.

Therefore, it was shown that Streptomyces has more than 10% heterogeneity compared with Micromonopora, which is the closest to Streptomyces in terms of phylogenetic relationship. Compared to similarity based on 16S rDNA nucleotide sequences, Micromonopora has a more than 95% identity with Streptomyces, so the 306-bp rpoB gene fragment of the present invention is appropriate for identifying Streptomyces species.

The homology between 161 Streptomyces reference strains except M. echinospora ranges from 99.7% to 88.9% (homology between S. armeniacus and S. lincolensis), so heterogeneity is 0.3 to 11.1% in the nucleotide sequences. Thus it is confirmed that interspecies variation is high compared to 16S rDNA in which the range of interspecies variation is not over 3%.

The present invention provides a method for identifying a rifampin-resistant Streptomyces and a rifampin-sensitive Streptomyces by using nucleotide sequence differences of rpoB genes which correspond to the 352 codon of the rpoB gene in S. coelicolor. The present invention uses the polynucleotide or parts thereof of an rpoB gene of Streptomyces, and as examples, the polynucleotide includes, but is not limited to, a nucleotide consisting of SEQ ID NO: 6 to SEQ ID NO: 167. The part of the 306-bp rpoB gene fragment of Streptomyces can be a 3 to 352-bp long nucleotide sequence comprising a nucleotide encoding the 352^{nd} amino acid of S. coelior corresponding to the 351^{st} amino acid of E. coli. The rpoB gene fragment can be prepared by amplifying the rpoB gene of Streptomyces with the primers specific for Streptomyces by a PCR.

The amino acid corresponds to the 531^{st} codon of the rpoB of E. coli. The rpoB fragment is prepared by a PCR with a primer specific to an rpoB gene of Streptomyces. Preferably, the nucleotide for distinguishing a rifampin genotype or a sensitive genotype is the 258-bp to 260-bp nucleotide sequence from the 5′-terminus of the 352-bp polynucleotide obtained by a PCR amplifying an rpoB gene of Streptomyces, and it is 234-bp to 236-bp in the case of a 306-bp fragment. If the nucleotide sequence of the strain is AAC encoding asparagine, the strain can be identified as a rifampin-resistant strain, and if the nucleotide sequence of the strain is a TCG or TCC encoding serine, the strain can be identified as a rifampin-sensitive strain.

An antibiotic which causes resistance via a single mechanism, and which contains a target gene involved in the resistance, can be useful for genotyping the bacteria. Although there are no studies on the mechanism of antibiotics resistance of Streptomyces, Mycobacterium tuberculosis, which is closest to Streptomyces in the phylogenetic tree, has only a resistance to rifampin caused by a genetic change in a single target gene. Thus, the resistance to rifampin is most useful in developing the screening method of the strain.

It has been reported that when the nucleotide sequence at the 531 codon of an rpoB gene fragment which corresponds to that of E. coli is mutated, resistance to a high concentration of rifampin is induced in E. coli, Mycobacterium tuberculosis, and Mycobacterium leprae (Singer M et al., J Mol. Biol. 5;231(1), pp. 1-5, 1993; Severinov K et al., Mol Gen Genet, 25, 244(2), pp. 120-126, 1994; Taniguchi H et al., FEMS Microbiol Lett. 15; 144(1), pp. 103-108, 1996). It has also been reported that B. burgdoferi which has been known to have a natural resistance to rifampin and T. pallidum, T. citri, etc. have AAC at the 531 codon of the rpoB gene (Aurivaud P et al., Antimicrob Agents Chemother. 1996; 40(4):858-62; Stamm L V et al., Antimicrob Agents Chemother. 2001 45(10):2973-4; Lee S H et al., J. Clin. Microbiol., 38(7):2557-2562, 2000).

In addition, the present invention is related to a primer specific to an rpoB gene of a rifampin-resistant or sensitive strain.

According to the present invention, a pair of primers which specifically amplify the rpoB gene of the rifampin-resistant Streptomyces comprise a nucleotide sequence consisting of SEQ ID NO: 3 as a forward primer, and a nucleotide sequence consisting of SEQ ID NO: 4 as a backward primer. The 243-bp nucleotide sequence comprising the primers which correspond to the 3^{rd} nucleotide of the 277 codon to the 2^{nd} nucleotide of the 358 codon in S. coelicolor can also be used for the present invention.

A pair of primers which specifically amplify the rpoB gene of the rifampin-sensitive Streptomyces comprise a nucleotide sequence consisting of SEQ ID NO: 3 as a forward primer, and a nucleotide sequence consisting of SEQ ID NO: 5 as a backward primer. The 243-bp nucleotide sequence comprising the primers which corresponds to the 3^{rd} nucleotide of the 277 codon to the 2^{nd} nucleotide of the 358 codon in S. coelicolor can also be used for the present invention.

The forward primer can be designed for amplifying all the Streptomyces species based on a region conserved in Streptomyces. For example, STRIF1 (5′-C GGC GAG CTS ATC CAG AAC C-3′) can be selected as a forward primer which is 20 base at the 3^{rd} nucleotide of the 277 codon to the 1^{st} nucleotide of the 284 codon in S. coelicolor (GenBank Accession No. AL160431.1). The STRIF1 is shown in SEQ ID NO: 3.

The backward primers can be designed for specifically amplifying and differentiating the rifampin-resistant strain and the rifampin-sensitive strain. The backward primer specific for the rifampin-resistant strain is designed to have GTT at its 3′-terminus, which is a complementary sequence of AAC of the 352 codon characterized in the rifampin-resistant strain. For example, S-AAC20 (5′-CC ACC CGG GCC SAG SGM GTT-3′) as set forth in SEQ ID NO: 4 is a 20 base sequence located in the 2^{nd} nucleotide of the 358 codon to the 1^{st} nucleotide of the 352 codon in the 3′ to 5′ direction. S-TCG20 is designed for only amplifying the rpoB gene of a rifampin-sensitive strain as a backward primer.

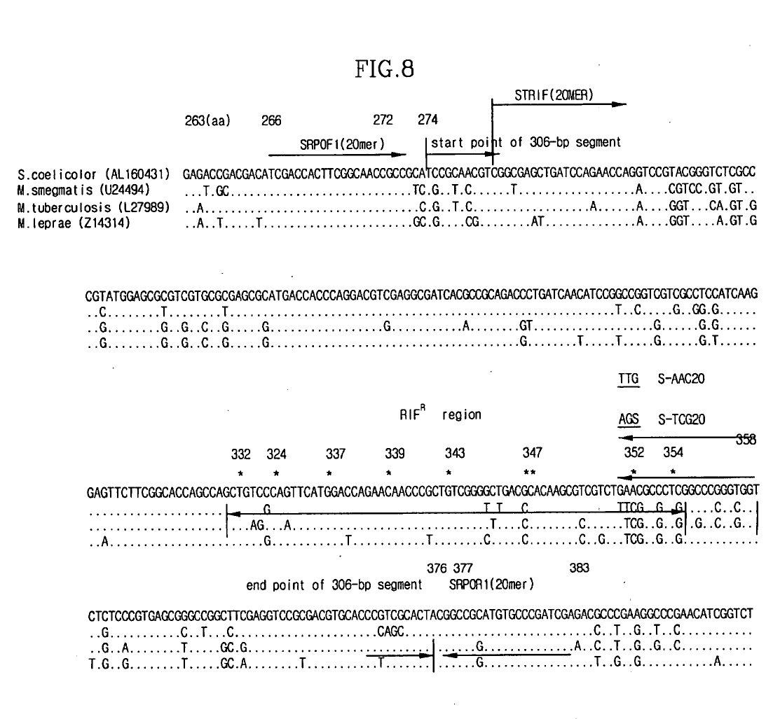

The rifampin-sensitive strain has TCG or TCC at the 352 codon of an rpoB gene. Thus, the backward primer specific for a rifampin-sensitive strain is different in 3 nucleotides at the 3′-terminus, compared with a primer specific to a rifampin-resistant strain, and thus the rpoB gene of the rifampin-sensitive strain cannot be amplified by using the backward primer of the rifampin-resistant strain. Like the backward primer specific for the rifampin-resistant strain, the backward primer specific for the rifampin-sensitive strain has a SGA 3′-terminus which complimentarily binds to TCG or TCC. Namely, S-TCG20 (5′-CC ACC CGG GCC VAG MGC SGA-3′) as set forth in SEQ ID NO: 5 is 20 base at the 2^{nd} nucleotide of the 358 codon to the 1^{st} nucleotide of the 352 codon in the 3′ to 5′ direction. In the nucleotide sequences of the primers, V, M, and S mean (G, A, C), (A, C), and (G, C) according to IUB code, respectively (FIG. 8).

FIG. 8 shows nucleotide sequences of primers which are specific for Streptomyces, a rifampin-sensitive Streptomyces, and a rifampin-resistant Streptomyces. The number in FIG. 8 indicates the number of base pairs of RNA polymerase beta-subunits of S. ceolicolor. The 69-bp long nucleotide sequences at the 332 to 354 positions are of an rifR region representing the rifampin resistance of Mycobacterium tuberculosis, which confirms that the 306-bp gene fragment of Streptomyces includes the rifR region. The asterisk represents a hot spot region where the nucleotide sequence is frequently mutated in Mycobacterium tuberculosis.

Also, the present invention provides identification of a rifampin-resistant strain or sensitive strain by using differences in nucleotide sequence coding of an rpoB amino acid of Streptomyces corresponding to the 352^{nd} amino acid of S. coelicolor rpoB.

The rifampin-resistant genotype or sensitive genotype were determined by amplifying an rpoB gene fragment containing an rpoB gene of Streptomyces corresponding to the 352^{nd} amino acid of the rpoB of S. coelicolor, by sequencing and by identifying whether nucleotide sequences of a region corresponding to the 352^{nd} amino acid is AAC encoding asparagine, or a TCT- or TCC-encoding serine. Preferably, nucleotide sequences for distinguishing rifampin-resistant genotypes or sensitive genotypes are sequences of 258-bp to 260-bp in the 5′ to 3′ direction among the 352-bp polynucleotide prepared by amplification with a primer set of SEQ ID NO: 1 and SEQ ID NO: 2, and they are sequences of 234-bp to 236-bp among the 306-polynucleotide.

Accordingly, any molecular biological method using nucleotide sequence differences as mentioned above can be applied to identification of the rifampin-resistant Streptomyces and the rifampin-sensitive Streptomyces. As examples, the methods include a sequencing method for rpoB, a PCR method that is capable of easily and rapidly identifying using a primer specific to the rifampin-resistant and rifampin-sensitive genotypes, a hybridization method for detecting rifampin-resistant and rifampin-sensitive genotypes by using rpoB gene fragments comprising the 352^{nd} codon region as a probe, and a microarray method of fixing probes comprising nucleotides coding the 352^{nd} amino acid of Streptomyces in a microarray and by contacting an amplified rpoB gene of sample strain thereto.

In an embodiment, the present invention provides a method for identifying the rifampin-resistant and rifampin-sensitive strains that comprises (a) amplifying an rpoB gene fragment of a strain of interest with a primer set specific to an rpoB gene fragment comprising nucleotides coding the 352^{nd} amino acid of an rpoB gene of Streptomyces; and (b) sequencing the nucleotide sequence coding the 352^{nd} amino acid in an amplified rpoB gene fragment. Preferably, the primer set comprises nucleotide sequences shown in SEQ ID NO: 1 and 2.

In an embodiment, the present invention that provides a method for identifying rifampin-resistant and rifampin-sensitive strains comprises (a) amplifying an rpoB gene fragment of a strain of interest in a sample with the primers for specifically amplifying rpoB genes of rifampin-resistant Streptomyces or rifampin-sensitive Streptomyces, and (b) analyzing whether the amplified product is produced or not.

The rpoB gene fragment of the target strain is amplified with a primer specific to the Streptomyces species by PCR, and nucleotide sequences are analyzed. PCR and nucleotide sequencing methods, which are known to an ordinary person skilled in the field, can be applied to the present invention (Kim B J, et al., J Clin Microbiol, 37(6), pp. 1714-20, 1999).

The primer set can be primer set selected from the group consisting of primer sets for specifically amplifying a rifampin-resistant Streptomyces of SEQ ID NO: 3 and SEQ ID NO: 4, primer sets for specifically amplifying a rifampin-sensitive Streptomyces of SEQ ID NO: 3 and SEQ ID NO: 5, and a mixture thereof. An example of the analyzing method is agarose gel or polyacrylamide gel electrophoresis, but it is not limited that.

The development of a molecular-biological screening method can be applied to selectively isolate and detect Streptomyces having a rifampin-resistant gene from soil or the ocean, in the future.

The present invention is further shown in the following examples, which should not be taken to limit the scope of the invention.

### EXAMPLE 1

### Preparation of rpoB Primer Specific to Streptomyces

Sequences of 4 kinds of microorganisms selected from GenBank were aligned, and sequences for specific primer regions were determined in order by the Genotech company.

For preparing primers capable of amplifying all kinds of Streptomyces, rpoB sequences of 4 kinds of Streptomyces including S. coelicolor (GenBank No. AL160431), M. smegmatis (GenBank No. U24494), M. tuberculosis (GenBank No. L27989), and M. leprae (GenBank No. Z14314) that are reported in GenBank were compared. The forward primer can be selected from a region which has 100% homology between 4 kinds of different genus strains, including 20 base (5′-TC GAC CAC TTC GGC AAC CGC-3′) located at the 2^{nd} nucleotide position of the 266 codon to the 3^{rd} nucleotide position of the 273 codon of S. coelicolor, and the forward primer is called SRPOF1 (FIG. 2).

The backward primer can be 20 base nucleotide sequences that have 100% nucleotide sequence homology with M. smegmatis, which belongs to the rapid-growing mycobacteria group but has have one different nucleotide compared with M. tuberculosis, and M. leprae, which belongs to the slow growing mycobacteria group (FIG. 2). The backward primer is called SRPOR1 (5′-TC GAT CGG GCA CAT GCG GCC-3′), and its nucleotide sequence is located at the 2^{nd} nucleotide position of the 383 codon to the 1^{st} nucleotide position of the 377 codon in the 3′ to 5′ direction in the rpoB gene of M. smegmatis.

### EXAMPLE 2

### Preparation of rpoB 306-bp Fragment of Streptomyces

2-1: Preparation of Strains

rpoB sequences of 163 kinds of reference strains including 161 strains of Streptomyces and a strain of micromonospora provided from the Korean Collection for Type Cultures of the Korea Research Institute of Bioscience and Biotechnology were analyzed (Tables 1a and 1b). A comparative sequence analysis for eight strains comprising 4 kinds of non-reference strains used for identification was carried out (Table 1).

2-2: DNA Isolation

DNA was prepared by the bead beater-phenol extraction (BB/P) method. A loop of culture of each isolate was suspended in TEN buffer (10 mM Tris-HCl, 1 mM EDTA, 100 mM NaCl; pH 8.0), placed in a tube filled with 100 μl (packed volume) of glass beads (diameter, 0.1 mm; Biospec Products, Bartlesville, Okla., U.S.A) and 100 μl of phenol:chloroform:isopropyl alcohol (50:49:1), and the tube was oscillated on a Mini-Bead Beater (Biospec Products) for 1 min to disrupt the bacteria. The disrupted bacteria was centrifuged at 12,000 rpm for 5 min and the supernatant (100 μl) was transferred into a new tube. 60 μl of isopropyl alcohol was then added thereto and it was centrifuged at 15,000 rpm for 15 min. The resulting pellet was washed with 70% ethanol, and a TE buffer (pH 8.0, 10 mM Tris-HCl, 1 mM EDTA) was added to obtain 60 μl of DNA.

2-3: Amplification of rpoB Gene by PCR

PCR reaction was carried out using AccuPower PCR PreMix (Korea, bioneer) containing 2 U Taq polymerase, 10 mM dNTP, 10 mM Tris-HCl (pH 8.3), 1.5 mM MgCl₂. Primer (Genotech) prepared by EXAMPLE 1 was used. 50 ng of each Streptomyces DNA as a template and 20 pmol of each primer, SRPOF1 and SRPOR1, were placed in a tube and distilled water was added thereto to a final volume of 20 μl. PCR was performed at 95° C. for 5 min for a first denaturation, followed by 30 cycles of 1 min at 95° C. for subsequent denaturation, 45 s at 62° C. for annealing, 1 min 30 s at 72° C. for extension, and 5 min at 72° C. for final extension (Model 9600 thermocycler, Perkin-Elmer cetus). After PCR, PCR products were electrophoresed on 1% agarose gel to observe a 352 bp fragment.

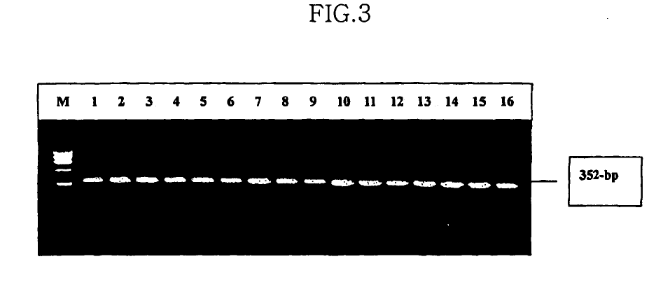

As a result of PCR using primer prepared by EXAMPLE 1, it was observed on the 1% agarose gel that all 162 reference strains were amplified as the rpoB DNA fragments of the 342 bp (FIG. 2). As well as Streptomyces, amplification was observed in Micromonospora sp. (FIG. 3, lane 3).

2-4: Isolation of PCR Products

After electrophoresis on 1% gel, a gel part containing the 352-bp of PCR product was cut and transferred into a new tube in order to isolate DNA. DNA isolation and purification were carried out using a Qiaex (Qiagen, Germany) system. The solution for gel dissolution QX1 500 μl was added to the tube, and the gel and solution were melted for 15 min at 50° C. Then, 10 μl of gel bead were mixed thereto and held at 50° C. for 15 min. The tube was subjected to a vortex for 10 s at intervals of 1 min to equally spread the beads. The tube contents were then washed once with QX1 and twice with QF, dried at 45° C. for 10 min, followed by addition of a TE buffer to obtain 20 μl of DNA.

### EXAMPLE 3

### Nucleotide Sequence Analysis of rpoB Fragment

The eluted DNA from the gel was used as a template, and automatic sequencing was performed. 60 ng of the template DNA, 1.2 pmol primer, 2 μl of dye from a BigDye Terminator Cycle Sequencing kit (PE Applied Biosystems) were mixed and distilled water were added thereto, to a final volume of 10 μl. Reaction was undertaken with a Perkin Elmer Cetus 9600 for 25 cycles of 10 s at 95° C., 10 s at 60° C., and 4 min at 60 s. DNA was purified from the reacted sample by an ethanol precipitation method. That is, after 180 μl of distilled water and 10 μl of 3 M sodium acetate were added to the sample to bring the total volume to 200 μl, twice the volume of 100% ethanol was mixed with the mixture and centrifuging was carried out to precipitate DNA. After adding 500 μl of 70% ethanol, centrifuging was carried out at 15,000 rpm for 20 min to wash the DNA. The DNA was recovered with formamide (PE Applied Biosystems).

The purified DNA was incubated at 95° C. for 5 min to generate single strand DNA, and the sequence was analyzed with an ABI 3100 system (ABI3100, PE Applied Biosystems) after electrophoresis for 2 hours 30 min. Sequence analysis was undertaken with forward primer SRPOF1 and backward primer SRPOR1 methods and a sequence of the 306-bp fragment except the primer region was determined to construct a database.

### EXAMPLE 4

### Alignment of rpoB Fragment (306 bp) Sequence, Analysis of Sequence Identity, and Construct of Phylogenetic Tree

The rpoB nucleotide sequence (306 bp) of 162 Streptomyces reference strains analyzed by EXAMPLE 3 were aligned by using the multiple alignment algorithm of the MegAlign package, and a database for rpoB of Streptomyces was constructed. For the multiple alignment, 306 bp nucleotides were translated to 161 amino acid residues and the amino acid residues were multiply aligned by a Clustal Method of the Megalign program. The database for identifying the Streptomyces was constructed using 306 bp nucleotides deduced from the aligned 161 amino acid residues.

Similarity among nucleotide sequences of 162 kinds of reference strains was analyzed using sequence distance measured within multiple alignment databases by the Megalign program. The phylogenetic relationship between strains was analyzed using a phylogenetic tree constructed by MEGA software (Kumar, S., K. Tamura, and N. Masatoshi. 1993. MEGA: molecular evolutionary genetics analysis, version 1.01. The Pennsylvania State University, University Park).

The multiple aligned 306-bp nucleotide from 102 kinds of strains was used to construct a Neighbor-joining phylogenetic tree based on the Juke-Cantor distance estimation method and a pairwise deletion method. An analysis of bootstrap was performed through 100 replications. As a result, the similarity of nucleotide sequences for the 306-bp fragment of rpoB and the phylogenetic tree are represented in FIG. 4 and FIG. 5.

### EXAMPLE 5

### Identification of Non-Reference Strains by the Comparative Sequence Analysis Using Database for the rpoB 306-bp Fragment of Reference

In order to determine whether a database for Streptomyces reference strains can be applied to identification of microorganisms or not, eight non-reference strains of four kinds, being 1 one strain of streptomyces olivichromogenes (KCTC9090); 2 strains of S. peucetius (KCTC 9038, KCTC 9242); 3 strains of S. hydroscopicus (KCTC 9030, KCTC 9031, KCTC 9069); and 2 strains of S. albus (KCTC 1136, KCTC 1533), were evaluated. Identification of microorganisms was carried out by comparative sequence analysis.

Firstly, DNA was extracted from each strain, and amplification of the rpoB gene and purification were carried out by same method as described in EXAMPLE 1. The 306-bp nucleotides of the purified products were then sequenced by the same method as described in EXAMPLE 2.

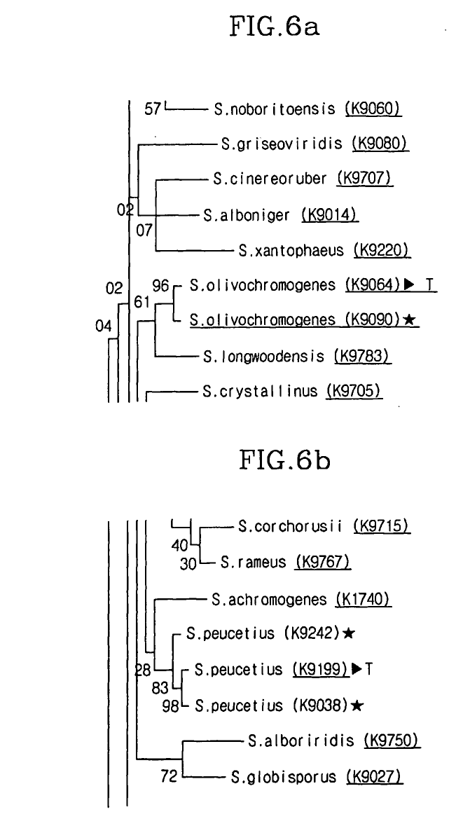

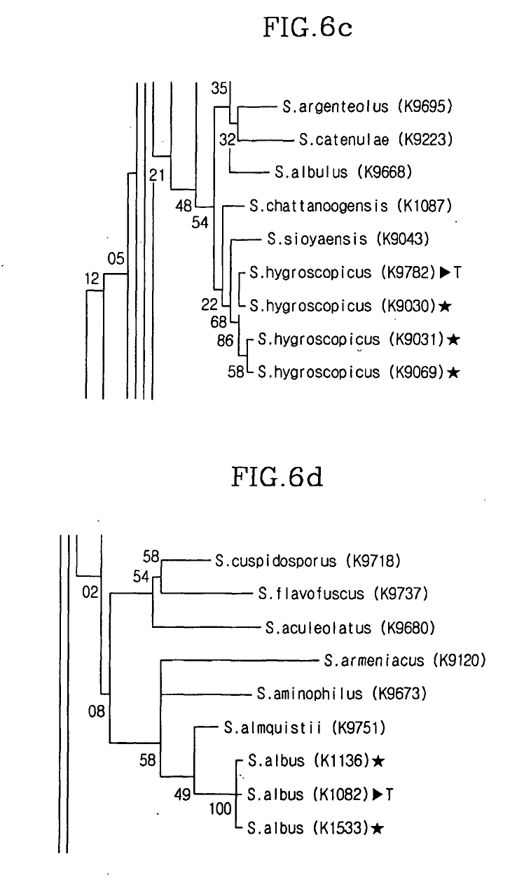

Each analyzed 306-bp nucleotide sequence was input into the Megalign program of Dnastar software in order to multiply align and develop the phylogenetic tree based on a Neighbor-Joining method of Mega software, and strains were identified. It was confirmed that one strain of streptomyces olivichromogenes (KCTC9090) as a non-reference strain showed 100% similarly to and was located at the streptomyces olivichromogenes (KCTC9064) loci reference strain in the phylogenetic tree (FIG. 6a); the two strains of S. peucetius (KCTC 9038, KCTC 9242) respectively had 100% and 99.7% nucleotide sequence homology with and were located at the same loci as the S. peucetius (KCTC 9199) reference strain in the phylogenetic tree (FIG. 6b); the three strains of S. hydroscopicus (KCTC 9030, KCTC 9031, KCTC 9069) respectively had 100%, 99.7%, and 99.7% of nucleotide sequence homology with and were located at the same loci as the S. hydroscopicus (KCTC 9782) reference strain in the phylogenetic tree (FIG. 6c); and both non-reference strains of S. albus (KCTC 1136, KCTC 1533) had 100% nucleotide sequence homology with and were located at the same loci of S. albus (KCTC 1082) as a reference strain in the phylogenetic tree (FIG. 6d).

### EXAMPLE 6

### Rifampin-Resistance Mechanism of Streptomyces

To determine whether AAC encoding Asp which is located at the 352^{nd} amino acid of the rpoB gene of S. coelicolor or at the 531^{st} amino acid of the rpoB gene of E. coli is related to a resistance to rifampin, a rifampin-sensitive test was carried out.

6-1: Strain Selection

A total of 47 strains of Streptomyces references including 24 reference strains having AAC encoding Asp and 23 reference strains having TCG or TCC encoding Ser at the sequenced positions were used, and 306-bI rpoB nucleotide sequences of the 48 strains are shown in SEQUENCE LISTING.

6-2: Verification of Rifampin-Resistance Mechanism of Streptomyces

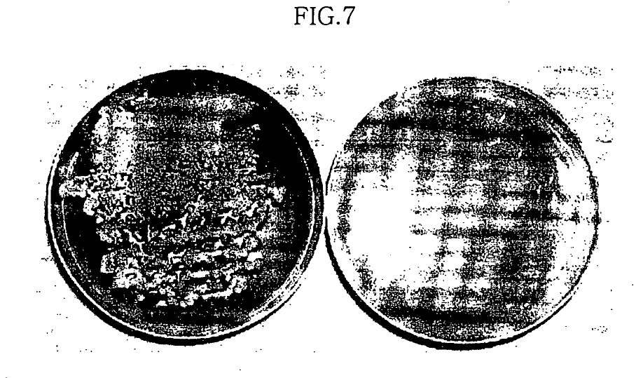

The 47 strains were respectively cultured at 28° C. for 72 hr in Bennet liquid media. The culture solution was then inoculated on Bennet solid media (yeast extract 1 g/L, Beef extract 1 g/L, Tryptone 2 g/L, Glycerol 10 g/L, Agar 15 g/L) containing 25 ug/ml rifampin, and after incubation for 72 hr, rifampin-resistance was tested. That is, a strain that generates colonies in solid media containing rifampin was identified as a positive strain for rifampin, while a strain that does not generate colonies was identified as a rifampin-sensitive strain, and the results are presented in FIG. 7 and Table 3.

TABLE 3

| | | | |
|---|---|---|---|
| No. | Strain | Source | genotype |

AAC genotype

| | | | |
|---|---|---|---|
| 1 | S. acrimycini | KCTC 9679 | + |
| 2 | S. aculeolatus | KCTC 9680 | + |
| 3 | S. alanosinicus | KCTC 9683 | + |
| 4 | S. aminophilus | KCTC 9673 | + |
| 5 | S. albogriseolus | KCTC 9773 | + |
| 6 | S. albus | KCTC 1082 | + |
| 7 | S. armeniacus | KCTC 9120 | + |
| 8 | S. avidinii | KCTC 9757 | + |
| 9 | S. capillispiralis | KCTC 1719 | + |
| 10 | S. cinerous | KCTC 9066 | + |
| 11 | S. coelicolor | KCTC 9005 | + |
| 12 | S. cuspidosporus | KCTC 9718 | + |
| 13 | S. durhamensis | KCTC 9723 | + |
| 14 | S. echinoruber | KCTC 9725 | + |
| 15 | S. ederensis | KCTC 9726 | + |
| 16 | S. flaveolus | KCTC 9022 | + |
| 17 | S. flavofuscus | KCTC 9737 | + |
| 18 | S. galilaeus | KCTC 1919 | + |
| 19 | S. griseus | KCTC 9080 | + |
| | griseus | | |
| 20 | S. phaeochromogenes | KCTC 9763 | + |
| 21 | S. plicatus | KCTC 9040 | + |
| 22 | S. pulveraceus | KCTC 9766 | + |
| 23 | S. sclerotialus | KCTC 9065 | + |
| 24 | S. spectabilis | KCTC 9218 | + |

TCG (TCC) genotype

| | | | |
|---|---|---|---|
| 25 | S. abikoensis | KCTC 9662 | − |
| 26 | S. achromogenes | KCTC 1740 | − |
| 27 | S. actuosus | KCTC 9112 | − |
| 28 | S. albireticuli | KCTC 9685 | − |
| 29 | S. albofaciens | KCTC 9747 | − |
| 30 | S. alboniger | KCTC 9014 | − |
| 31 | S. alboviridis | KCTC 9750 | − |
| 32 | S. albulus | KCTC 9668 | − |
| 33 | S. almquistii | KCTC 9751 | − |
| 34 | S. antimycoticus | KCTC 9694 | − |
| 35 | S. argenteolus | KCTC 9695 | − |
| 36 | S. bacillaris | KCTC 9018 | − |
| 37 | S. bambergiensis | KCTC 9019 | − |
| 38 | S. bikiniensis | KCTC 9172 | − |
| 39 | S. cacoi asoensis | KCTC 9700 | − |
| 40 | S. carpinensis | KCTC 9128 | − |
| 41 | S. catenulae | KCTC 9223 | − |
| 42 | S. celluloflavus | KCTC 9702 | − |
| 43 | S. chartreuses | KCTC 9704 | − |
| 44 | S. chattanoogensis | KCTC 1087 | − |
| 45 | S. chrysomallus | KCTC 9705 | − |
| 46 | S. cinereoruber | KCTC 9707 | − |
| 47 | S. cinnamonensis | KCTC 9708 | − |

Table 3 shows a relationship between the rpoB genotype and the rifampin phenotypes that are resistant or sensitive to rifampin, and the genotype was analyzed according to the rpoB nucleotides of 162 reference strains. In Table 3, the AAC genotype indicates Streptomyces that have AAC encoding asparagine corresponding to the 531^{st} amino acid of E. coli in nucleotide sequences of the rpoB gene, and the TCG (or TCC) genotype indicates strains that have TCG or TCC encoding serine in the same sequences. In the above Table, phenotype was determined according to grow strain in media containing rifampin or not, and “(+)” indicates a genotype with rifampin-resistance while “(−)” indicates a genotype with rifampin-susceptibility.

All 24 strains having AAC genotypes based on nucleotide sequence analysis were identified as rifampin-resistant strains, and the 23 strains having TCG or TCC genotypes encoding serine based on nucleotide sequence analysis were identified as rifampin-sensitive strains (FIG. 7 and Table 3).

FIG. 7 shows the strains cultured in Bennet solid media after S. cinerous (KCTC 9066) which is a rifampin-resistant strain having AAC encoding asparagine at the nucleotide sequence corresponding to the 531^{st} amino acid residue of the rpoB gene (A), and S. alboviridis (KCTC 9750) which is a rifampin-sensitive strain having TCG encoding serine (B) was cultured in Bennet liquid media. Thus the S. cinerous (KCTC 9066), a rifampin-resistant strain, generated colonies in media containing rifampin, while S. alboviridis (KCTC 9750), a rifampin-sensitive strain, did not show growth in the same media.

### EXAMPLE 7

### Primer Specific to Rifampin-Resistant Strain

In order to amplify rifampin-resistant strains, STRIF1 and SAAC20 which can amplify a 243-bp of a rpoB gene fragment comprising from the 3^{rd} nucleotide of the 277 codon to the 2^{nd} nucleotide of 358-bp in S. coelicolor were used. As shown in FIG. 8, a backward primer, S-AAC20, is specific to the rifampin-resistant strain. Thus, the former has GTT, a reverse form of AAC, at the 3′-terminus of the primer and generates a PCR product specific to a rifampin-resistant strain.

Among an rpoB full sequence (GenBank No. AL160431.1) of Streptomyces coelicolor, 20mer of STRIF1 (5′-C GGC GAG CTS ATC CAG AAC C-3) comprising nucleotides from the 3^{rd} nucleotide of the 277 codon to the 1^{st} nucleotide of the 248 codon was selected as a forward primer.

A backward primer specific to a rifampin-resistant strain and a backward primer specific to a rifampin-sensitive strain were respectively prepared. Thus, a primer specific to a rifampin-resistant strain has GTT which is a reverse form of AAC encoding the 352^{nd} amino acid that is a characteristic codon in a rifampin-resistant strain, at the 3′-terminus of the primer. Therefore, S-AAC20 (5′-CC ACC CGG GCC SAG SGM GTT-3) comprising 20mer of nucleotides from the 2^{nd} nucleotide of the 358 codon to the 1^{st} nucleotide of the 352 codon was selected as a backward primer for specifically amplifying a rifampin-resistant strain.

### EXAMPLE 8

### Primer Specific to Rifampin-Sensitive Strain

In order to amplify rifampin-sensitive strains, STRIF1 and S-TGC20 primer sets which can amplify a 243-bp of an rpoB gene fragment comprising from the 3^{rd} nucleotide of the 277 codon to the 2^{nd} nucleotide of the 358 codon in S. coelicolor were used. As shown in FIG. 8, the backward primer, S-TCG20, is specific to rifampin-sensitive strains, so it generates a PCR product specific to rifampin-sensitive strains.

The backward primer specific to rifampin-sensitive strains has SGA, which is the reverse of TCG or TCC, and it is a characteristic codon in a rifampin-sensitive strain, at the 3′-terminus. Therefore, S-TCG20 (5′-CC ACC CGG GCC VAG MGC SGA-3′) comprising 20mer of nucleotides from the 2^{nd} nucleotide of the 358 codon to the 1^{st} nucleotide of the 352 codon was selected as the backward primer for specifically amplifying rifampin-sensitive strains. “V”, “M”, and “S” among the primer sequence mean (G, A, C), (A, C), and (G, C) according to the IUB code (FIG. 8).

### EXAMPLE 9

### Detection of Rifampin-Sensitive Strain or Rifampin-Resistant Strain by Using Method of Analyzing Nucleotide Sequence of rpoB Gene

The primer sets of SEQ ID NOs: 1 and 2 were used to amplify rpoB genes of 60 strains, and nucleotides of the 352-bp products were analyzed with an automatic sequencer. SRPOF1 as a forward primer and SRPOR1 as a backward primer were used.

FIG. 8 is an electropherogram obtained by amplifying 306-bp rpoB gene fragment with the SRPOF1 and SRPOR1 primer set and automatically sequencing with an SRPOF1 primer. (A) is an rpoB nucleotide sequence of S. anthocyanicus (KCTC 9755), and it is confirmed that AAC nucleotides exist at the 352^{nd} amino acid on the basis of S. coelicolor. (B) is an rpoB nucleotide sequence of S. humidus (KCCM 40647), and it is confirmed that TCG nucleotides encode serine which show a rifampin-sensitive genotype. (C) is an rpoB nucleotide sequence of S. atroolvaceous (KCTC9017), and it is confirmed that TCG nucleotides encode serine which shows a rifampin-sensitive genotype. And, these genotypes are identical to the known sensitivity results. That is, S. anthocyanicus (KCTC 9755) having a rifampin-resistant AAC genotype was determined to be a rifampin-resistant strain, and S. humidus (KCCM 40647) and S. atroolvaceous (KCTC9017) having TGC or TCC genotypes were determined to be rifampin-sensitive strains.

When sequences of 60 strains were analyzed and genotypes were determined according to the method mentioned above, 19 strains were identified as rifampin negative. That is, the 19 strains had AAC at the 352^{nd} position of the amino acid on the basis of the rpoB gene of S. coelicolor. The other 41 strains were identified as rifampin-sensitive genotypes. That is, TCG or TCC was located at the 352^{nd} position. These results shown 100% sensitivity and specificity, and were identical to the established result for rifampin susceptibility (Table 4). “(+)” is a rifampin-resistant strain and “(−)” is a rifampin-sensitive strain in the below Table 4.

TABLE 4

| | | | |
|---|---|---|---|
| No. | Name | Source | genotype |

AAC genotype

| | | | |
|---|---|---|---|
| 1 | S. albaduncus | KCTC 1741 | + |
| 2 | S. althioticus | KCTC 9752 | + |
| 3 | S. ambofaciens | KCCM 40182 | + |
| 4 | S. anulatus | KCCM 40190 | + |
| 5 | S. anthocyanicus | KCTC 9755 | + |
| 6 | S. cellulose | KCTC 9703 | + |
| 7 | S. chivaensis | KCTC 9786 | + |
| 8 | S. coelescens | KCCM 40742 | + |
| 9 | S. griseoflavus | KCCM 12624 | + |
| 10 | S. humiferus | KCTC 9116 | + |
| 11 | S. lividans | KCTC 1154 | + |
| 12 | S. murinus | KCTC 9492 | + |
| 13 | S. piosus | KCCM 40480 | + |
| 14 | S. rubiginosus | KCTC 9042 | + |
| 15 | S. tendae | KCCM 40105 | + |
| 16 | S. umbrinus | KCCM 40316 | + |
| 17 | S. violaceoruber | KCTC 9787 | + |
| 18 | S. xanthocidicus | KCCM 40286 | + |
| 19 | S. yokosukanens | KCCM 40633 | + |

TCG (TCC) genotype

| | | | |
|---|---|---|---|
| 20 | S. amakusaensis | KCTC 9753 | − |
| 21 | S. aburaviensis | KCTC 9663 | − |
| 22 | S. albospinus | KCTC 9762 | − |
| 23 | S. albovinaceous | KCCM 40177 | − |
| 24 | S. anabdii | KCTC 9687 | − |
| 25 | S. antibioticus | KCTC 1137 | − |
| 26 | S. atroolvaceous | KCTC 9017 | − |
| 27 | S. aureufaciens | KCCM 40127 | − |
| 28 | S. azureus | KCCM 40485 | − |
| 29 | S. baldacii | KCCM 41326 | − |
| 30 | S. candidus | KCTC 9020 | − |
| 31 | S. caseius | KCCM 40740 | − |
| 32 | S. californicus | KCCM 40605 | − |
| 33 | S. carpinensis | KCTC 9128 | − |
| 34 | S. chromogenes | KCCM 40727 | − |
| 35 | S. cinnamoneus | KCCM 40572 | − |
| 36 | S. citreofluorescens | KCTC 9710 | − |
| 37 | S. coerulescens | KCCM 40508 | − |
| 38 | S. coeruleofuscus | KCCM 40506 | − |
| 39 | S. coralus | KCCM 40642 | − |
| 40 | S. cremeus | KCCM 40509 | − |
| 41 | S. cyaneofuscatus | KCCM 40517 | − |
| 42 | S. disatochromogenes | KCCM 40449 | − |
| 43 | S. erumpens | KCTC 9729 | − |
| 44 | S. erythraeus | KCCM 40477 | − |
| 45 | S. eurythermus | KCTC 9731 | − |
| 46 | S. fimbriatus | KCCM 11888 | − |
| 47 | S. flavotricini | KCCM 40520 | − |
| 48 | S. flavovirens | KCCM 40165 | − |
| 49 | S. fulvissimus | KCTC 9773 | − |
| 50 | S. fumanus | KCCM 40522 | − |
| 51 | S. gougeroti | KCCM 40681 | − |
| 52 | S. griseoruber | KCCM 40658 | − |
| 53 | S. griseolosporeus | KCTC 9791 | − |
| 54 | S. griseostramineus | KCCM 40526 | − |
| 55 | S. hachijoense | KCCM 32306 | − |
| 56 | S. halstedii | KCCM 40613 | − |
| 57 | S. humidus | KCCM 40647 | − |
| 58 | S. indigoferus | KCCM 40495 | − |
| 59 | S. kifunensis | KCTC 9734 | − |
| 60 | S. kurssanovi | KCCM 40527 | − |

### EXAMPLE 10

### Detection of Rifampin-Resistant Strain or Rifampin-Sensitive Strain by PCR

The inventors developed a PCR method for specifically amplifying rifampin-resistant strains and rifampin-sensitive strains using the three primers (STRI-F, S-AAC20, S-TCG20) prepared in EXAMPLE 7 and 8.

10-1: Specific Amplification of Rifampin-Resistant Strain

For a total of 47 Streptomyces strains comprising 24 strains of rifampin-resistant AAC genotypes and 23 strains of rifampin-sensitive genotypes selected from the Table 3, PCR was carried out with a forward primer, SRPOF1 which was specific to rifampin-resistant Streptomyces, and a backward primer, S-AAC20, and then a 243-bp rpoB PCR product specific to rifampin-resistant strain was observed.

PCR reaction was carried out using an AccuPower PCR PreMix (Bioneer, Korea) including 2 U of Taq polymerase, 10 mM dNTP, 10 mM Tris-HCl (pH 8.3), and 1.5 mM MgCl₂. 50 ng template DNA, 20 pmol SRPOF1 primer, and 20 pmol S-AAC20 primer and distilled water to bring the total to 20 μl were mixed. PCR was run through a 1^{st} denaturation for 5 min at 95° C., and 35 cycles comprising denaturation for 1 min at 95° C., annealing for 45 s at 64° C., extension for 1 min 30 s at 72° C., and a final extension for 5 min at 72° C. (Model 9600 thermocycler, Perkin-Elmer cetus). After PCR, a 243-bp PCR product was observed by electrophoresis on 1.5% agarose gel.

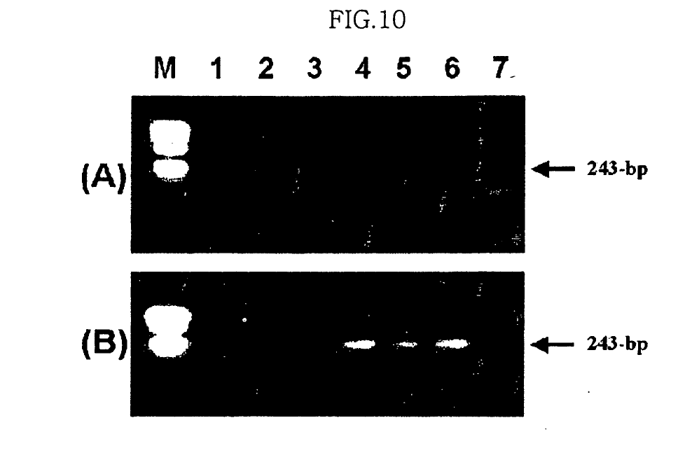

FIG. 10 is a photograph showing the results of identifying a rifampin-resistant strain and a rifampin-sensitive strain by using the PCR system of the present invention. In FIG. 10, lane M is a DNA size marker 174/Hae-III ladder, and lanes 1 to 7 are PCR results, wherein lane 1 is S. acrimycini (KCTC 9679), lane 2 is S. albus (KCTC 1082), lane 3 is S. cinerous (KCTC 9066), lane 4 is S. alboviridis (KCTC 9750), lane 5 is S. bacillaris (KCTC 9018), lane 6 is S. bikiniensis (KCTC 9172), and lane 7 is S. cinnamonensis (KCTC 9708).

In PCR using STRI-F and S-AAC20, which are specific to rifampin-resistant strains, 24 strains with rifampin-resistant AAC genotypes were amplified while 23 strains with TCG genotypes were not amplified.

10-2: Specific Amplification of Rifampin-Sensitive Strain

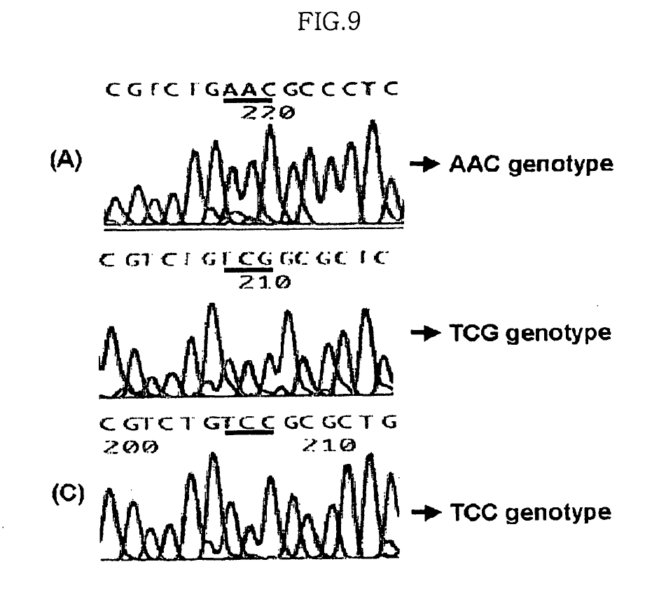

For a total of 47 Streptomyces strains comprising 24 strains of rifampin resistant AAC genotypes and 23 strains of rifampin sensitive genotypes selected from the Table 3, PCR was carried out with a forward primer, STRIF, and a backward primer, S-TCG20, by the same method described in EXAMPLE 10-1, and then rpoB PCR products specific to rifampin-sensitive strains were observed. The results are presented in FIG. 9.

In contrast to the rifampin-resistant strains, the 23 rifampin-sensitive strains were specifically amplified while the 24 rifampin-resistant strains were not amplified in PCR using STRI-F and S-AAC20, which are specific to rifampin-sensitive strains.

Accordingly, a simple and novel PCR method for screening rifampin-resistant strains was established. Strains that can generate a 243-bp gene fragment amplified by a STRIF and S-AAC20 primer set but cannot generate the fragment amplified by a STRIF and S-TCG20 primer set are identified as rifampin-resistant Streptomyces. Contrarily, strains that can generate a 243-bp gene fragment amplified by a STRIF and S-TCG20 primer set but cannot generate the fragment amplified by a STRIF and S-AAC20 primer set are identified as rifampin-sensitive Streptomyces.

10-3: Specific Amplification of Rifampin-Resistant and Sensitive Strain

For a total of 60 Streptomyces strains as shown in Table 4, PCR was carried out with each primer specific to rifampin-resistant strains or rifampin-sensitive strains which were prepared in EXAMPLEs 7 and 8 according to the method described in EXAMPLE 10-1, and it was shown that 19 strains with AAC genotypes were only amplified by a PCR specific to rifampin-resistant strains, but they were not amplified by a PCR specific for rifampin-sensitive strains. 41 strains with TCG or TCC genotypes were amplified to 243-bp gene fragments by a PCR specific to rifampin-sensitive strains but they were not amplified by a PCR specific to rifampin-resistant strains.

As shown above, the PCR method of the present invention shows results corresponding to the reported results about nucleotide sequences and sensitivity. Thus the identification method of rifampin-resistant or sensitive strains by using differences in nucleotide sequences corresponding to the 352^{nd} codon of the rpoB gene is very effective in terms of time and cost compared to the known detection method, and it can be applied to determine rifampin genotypes.

The present invention provides a polynucleotide of the 306 fragment of the RNA polymerase (rpoB) gene and an identification method of the Streptomyces rpoB genotype using the same, and thereby the present invention can be applied to identification or detection of Streptomyces strains due to improving on problems of slow growth, various strains, and material-centered identification, and it provides an easy, economical, and accurate identifying or detecting method. In addition, the identifying method of rifampin-resistant or sensitive strains by using differences in specific nucleotide sequences of rpoB genes has advantages in terms of efficiency in cost and time compared to the known detection method, and it can be widely used for identifying rifampin genotypes in the future.

1. A polynucleotide which is a 306-bp fragment of RNA polymerase-subunit (rpoB) of Streptomyces, and which is selected from the group consisting of nucleotide sequences set forth in SEQ ID NO: 6 to SEQ ID NO: 167.

2. A method for identifying Streptomyces species by using an rpoB gene fragment which comprises the steps of:
(1) amplifying an rpoB gene fragment of a strain of interest in a sample with primers for specifically amplifying rpoB genes of Streptomyces;
(2) analyzing a nucleotide sequence of the amplified rpoB gene fragment; and
(3) comparing the nucleotide sequence obtained in step (2) with rpoB 306-bp fragments of reference strains, wherein the rpoB 306-bp fragment of the reference strain is at least one selected from the group consisting of the nucleotide sequences set forth in SEQ ID NO: 6 to SEQ ID NO: 167.

3. The method of claim 2, wherein the primers have nucleotide sequences set forth in SEQ ID NO: 1 and SEQ ID NO: 2.

4. (canceled)

5. The method of claim 2, wherein in step (3), the nucleotide sequence homology of rpoB gene fragments between the strain of interest and the reference strain is at least 99.7%.

6. The method of claim 2, wherein in step (3), the comparing of the nucleotide sequence is performed by multiply aligning the nucleotide sequence of the rpoB gene fragment of the strain of interest with the nucleotide sequence of the rpoB gene fragment of the reference strain, by preparing a phylogenetic tree and by identifying the strain of interest.

7. A method for identifying a rifampin-resistant and a rifampin-sensitive Streptomyces which comprises analyzing differences in nucleotide sequences encoding the amino acid of an rpoB protein wherein the amino acid corresponds to the 352^{nd} amino acid of the RNA polymerase subunit B (rpoB) of Streptomyces coelicolor.

8. The method of claim 7, wherein the nucleotide sequence encoding the amino acid is a nucleotide sequence located at the 259-bp to 260-bp positions from the 5′-terminal of a 352-bp fragment prepared by amplifying an rpoB gene of Streptomyces with primers set forth in SEQ ID NOs: 1 and 2.

9. The method of claim 7, wherein the method comprises the steps of:
(a) amplifying the rpoB gene fragment of the strain of interest in a sample with primers set forth in SEQ ID NO: 1 and SEQ ID NO: 2 to produce a 352-bp polynucleotide; and
(b) analyzing the nucleotide sequence located at the 259 bp to 260-bp positions from the 5′-terminal of the polynucleotide.

10. The method of claim 7, wherein the nucleotide sequence encoding the amino acid is AAC for a rifampin-resistant strain.

11. The method of claim 7, wherein the nucleotide sequence encoding the amino acid is TCG or TCC for a rifampin-sensitive strain.

12. The method of claim 7, wherein the method comprises the steps of:
(a) amplifying an rpoB gene fragment of a strain of interest in a sample with primers for specifically amplifying rpoB genes of rifampin-resistant Streptomyces or rifampin-sensitive Streptomyces; and
(b) analyzing whether the amplified product is produced or not.

13. The method of claim 12, wherein the primers are selected from the group consisting of primers for rifampin-resistant Streptomyces set forth in SEQ ID NO: 3 and 4, primers for rifampin-sensitive Streptomyces set forth in SEQ ID NO: 3 and 5, and a mixture thereof.

14. A primer for specifically amplifying an RNA polymerase B subunit (rpoB) gene of the Streptomyces species, which is at least one selected from the group consisting of SEQ ID NOs: 1 and 2.

15. A primer set for specifically amplifying RNA polymerase B subunit (rpoB) genes of a rifampin-resistant Streptomyces, which comprises a forward primer comprising a nucleotide sequence set forth in SEQ ID NO: 3, and a backward primer comprising a nucleotide sequence set forth in SEQ ID NO: 4.

16. A primer set for specifically amplifying RNA polymerase B subunit (rpoB) genes of a rifampin-sensitive Streptomyces, which comprises a forward primer comprising a nucleotide sequence set forth in SEQ ID NO: 3, and a backward primer comprising a nucleotide sequence set forth in SEQ ID NO: 5.

17. The method of claim 8, wherein the nucleotide sequence encoding the amino acid is AAC for a rifampin-resistant strain.

18. The method of claim 9, wherein the nucleotide sequence encoding the amino acid is AAC for a rifampin-resistant strain.

19. The method of claim 8, wherein the nucleotide sequence encoding the amino acid is TCG or TCC for a rifampin-sensitive strain.

20. The method of claim 9, wherein the nucleotide sequence encoding the amino acid is TCG or TCC for a rifampin-sensitive strain.


SEQUENCE LISTING (167 sequences)

SEQ ID NO: 1 - DNA, 20 bp, Artificial Sequence
  forward primer(SRPOF1)
tcgaccacttcggcaaccgc

SEQ ID NO: 2 - DNA, 20 bp, Artificial Sequence
  reverse primer(SRPOR1)
tcgatcgggcacatgcggcc

SEQ ID NO: 3 - DNA, 20 bp, Artificial Sequence
  primer(STRIF)
cggcgagctsatccagaacc

SEQ ID NO: 4 - DNA, 20 bp, Artificial Sequence
  primer(S-AAC20)
ccacccgggccsagsgmgtt

SEQ ID NO: 5 - DNA, 20 bp, Artificial Sequence
  primer(S-TCG20)
ccacccgggccvagmgcsga

SEQ ID NO: 6 - DNA, 306 bp, K.azatica
tgcgcaacgtcggcgagctcatccagaaccaggtccgtaccggcctggcccggatggagcgcgtcgtccgcgagcggatgaccacgcaggacgtcgaggcgatcacgccgcagaccctgatcaacatccggccggtcgtcgcctccatcaaggagttcttcggcaccagccagctgtcccagttcatggaccagacgaacccgctgtcgggcctgacccacaagcgtcgtctgtccgcgctgggccccggtggtctgtcccgtgagcgggccggcttcgaggtccgtgacgtgcacccctcgcact

SEQ ID NO: 7 - DNA, 306 bp, K. cystarginea
tgcgcaacgtcggcgagctgatccagaaccaggtccgtacgggtctcgcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcgcagttcatggaccagacgaacccgctgtcgggcctgacccacaagcgccgtctgtccgccctcggtcccggtggtctctcccgtgagcgcgccggcttcgaggtccgtgacgtgcacccctcgcact

SEQ ID NO: 8 - DNA, 306 bp, K. griseola
tgcgcaacgtcggcgagctcatccagaaccaggtccgcaccggcctggcccgcatggagcgcgtcgtccgcgagcggatgaccacccaggacgtcgaggcgatcacgccgcagaccctgatcaacatccggccggtcgtcgcctccatcaaggagttcttcggcaccagccagctgtcccagttcatggaccagacgaacccgctgtcgggcctgacccacaagcgccgtctgtccgcgctgggccccggcggtctctcccgtgagcgggcgggcttcgaggtccgtgacgtgcacccctcgcact

SEQ ID NO: 9 - DNA, 306 bp, K. mediocidica
tgcgcaacgtcggcgagctgatccagaaccaggtccgtacgggtctcgcccgtatggagcgcgtcgtgcgcgagcgcatgaccacccaggacgtcgaggcgatcacgccgcagaccctgatcaacatccggccggtcgtcgcctccatcaaggagttcttcggtaccagccagctgtcccagttcatggaccagacgaacccgctgtcgggcctgacccacaagcgtcgtctgtccgcgctgggcccgggtggtctgtcccgtgagcgcgccggcttcgaggtccgtgacgttcacccgtcgcact

SEQ ID NO: 10 - DNA, 306 bp, K. phosalacinea
tgcgcaacgtcggcgagctgatccagaaccaggtccgcaccggcctcgcccgcatggagcgagtggtccgcgagcgcatgaccacccaggacgtcgaggcgatcacgccgcagaccctgatcaacatccggccggtcgtcgcctccatcaaggagttcttcggcaccagccagctgtcgcagttcatggaccagacgaacccgctgtcgggcctgacccacaagcgtcgtctgtccgcgctcggccccggcggtctgtcccgtgagcgggccggcttcgaggtccgagacgtgcacccctcgcact

SEQ ID NO: 11 - DNA, 306 bp, K. setae
tgcgcaacgtcggcgagctcatccagaaccaggtccgcaccggcctggcccggatggagcgcgtcgtccgcgagcggatgaccacccaggacgtcgaggcgatcacgccgcagaccctgatcaacatccggccggtcgtcgcctccatcaaggagttcttcggcaccagccagctgtcgcagttcatggaccagacgaacccgctgtcgggcctgacccacaagcgccgtctgtccgcgctcggccccggtggtctgtcccgcgagcgggccggcttcgaggtccgtgacgtgcacccctcgcact

SEQ ID NO: 12 - DNA, 306 bp, M. echinospora
tgcgtaccgtcggcgagctgatccagaaccaggtccgggtcggcctctcccgcatggagcgggtcgtccgcgagcggatgaccacccaggacgtcgaggcgatcacgccgcagaccctgatcaacatccgcccggtggtggcggcgatcaaggagttcttcggcacgtcgcagctgtcccagttcatggaccagaccaacccgctggcgggcctgacccaccggcgccggctgagcgcgctcggcccgggtggtctgtcccgggagcgggccggcttcgaggtccgggacgtgcacccgtcgcact

SEQ ID NO: 13 - DNA, 306 bp, S. abikoensis
tgcgcaacgtcggcgagctcatccagaaccaggtccgcacgggtctggctcgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaccaacccgctgtcgggtctgacccacaagcgtcgtctgtcggcgctgggtccgggcggtctctcccgtgagcgggccggcttcgaggtccgagacgtgcacccgtcgcact

SEQ ID NO: 14 - DNA, 306 bp, S. achromogenes
tgcgcaacgtcggcgagctgatccagaaccaggtccctacgggtctggcccgtatggagcgcgtcgtgcgcgagcgcatgacgacccaggacgtcgaggcgatcacgccgcagaccctgatcaacatccggccggtcgtcgcctccatcaaggagttcttcggcaccagccagctgtcgcagttcatggaccagaacaacccgctgtcgggtctcacccacaagcgccgtctgtcggctcttggtccgggtggtctctcccgtgagcgggccggcttcgaggtccgtgacgtgcacccctcgcact

SEQ ID NO: 15 - DNA, 306 bp, S. acrimycini
tccgcaacgtcggcgagctgatccagaaccaggtccgtacgggtctcgcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcgcagttcatggaccagaacaacccgctgtcggggctgacgcacaagcgtcgtctgaacgccctcggcccgggtggtctctcccgtgagcgggccggcttcgaggtccgtgacgtgcacccctcgcact

SEQ ID NO: 16 - DNA, 306 bp, S. actuosus
tccgcaacgtcggcgagctgatccagaaccaggtccgcacgggtctcgcccgtatggagcgtgtcgtccgcgagcggatgacgacccaggacgtcgaggcgatcacgccgcagaccctgatcaacatccggccggtcgtcgcctccatcaaggagttcttcggcaccagccagctgtcccagttcatggaccagaacaacccgctgtccgggctgacgcacaagcgtcgtctgtccgcgctcggcccgggtggtctctcccgtgagcgggccggcttcgaggtccgtgacgtccacccgtcgcact

SEQ ID NO: 17 - DNA, 306 bp, S. aculeolatus
tccgcaacgtcggcgagctgatccagaaccaggtgcgtacgggcctcgcccgtatggagcgcgtcgtgcgcgagcgcatgacgacccaggacgtcgaggcgatcacgccgcagaccctgatcaacatccggccggtcgtcgcctccatcaaggagttcttcggcaccagccagctgtcgcagttcatggaccagacgaacccgctgtccggtctgacccacaagcgccggctcaacgcgctcggccccggtggtctgtcccgtgagcgggccggcttcgaggtccgcgacgtgcacccgtcgcact

SEQ ID NO: 18 - DNA, 306 bp, S. alanosinicus
tccgcaacgtcggcgagctgatccagaaccaggtgcgtacgggtctcgcccgtatggagcgcgtcgtccgcgagcgcatgaccacccaggacgtcgaggcgatcacgccgcagacgctgatcaacatccggccggtcgtcgcctccatcaaggagttcttcggcaccagccagctgtcccagttcatggaccagaacaacccgctgtcggggctgacgcacaagcgtcgtctgaacgccctcggcccgggtggtctctcccgtgagcgggccggcttcgaggtccgtgacgtgcacccctcgcact

SEQ ID NO: 19 - DNA, 306 bp, S. albireticuli
tgcgcaacgtcggcgagctcatccagaaccaggtccgcacgggtctggctcgtatggagcgcgtcgtccgcgagcgcatgaccacgcaggacgtcgaggcgatcacgccgcagaccctgatcaacatccggccggtcgtcgcctccatcaaggagttcttcggcaccagccagctgtcccagttcatggaccagacgaacccgctctcgggtctgacgcacaagcgtcgtctgtccgcgctgggcccgggtggtctgtcccgtgagcgggccggcttcgaggtccgagacgtccacccgtcgcact

SEQ ID NO: 20 - DNA, 306 bp, S. albofaciens
tgcgcaacgtcggcgagctgatccagaaccaggtccgtacgggtctcgcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaccaacccgctgtcgggcctgacccacaagcgccgtctgtcggcgctgggccccggtggtctctcccgtgagcgggccggcctggacgtccgtgacgtgcacccctcgcact

SEQ ID NO: 21 - DNA, 306 bp, S. alboflavus
tgcgcaacgtcggcgagctcatccagaaccaggtccgcacgggtctggctcgtatggagcgcgtcgtgcgtgagcgcatgacgacgcaggacgtcgaggcgatcacgccgcagacgctgatcaacatccggccggtcgtcgcctccatcaaggagttcttcggcaccagccagctgtcgcagttcatggaccagaacaacccgctgtcgggtctcacccacaagcgccgactgtcggctcttggcccgggtggtctctcccgtgagcgggccggcttcgaggttcgtgacgtgcacccgtcgcact

SEQ ID NO: 22 - DNA, 306 bp, S. albogriseolus
tccgcaacgtcggcgagctgatccagaaccaggtccgtacgggtctcgcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcggggctgacgcacaagcgtcgtctgaacgccctcggcccgggtggtctctcccgtgagcgggccggcttcgaggtccgtgacgtgcacccctcgcact

SEQ ID NO: 23 - DNA, 306 bp, S. albolongus
tgcgcagcgtgggcgagctcatccagaaccaggtccgcaccggcctggcccgtatggagcgcgtcgtccgcgagcgcatgacgacccaggacgtcgaggcgatcacgccgcagaccctgatcaacatccggccggtcgtcgcctccatcaaggagttcttcggcaccagccagctgtcccagttcatggaccagaacaacccgctgtccgggctgacgcacaagcggcgtttgtccgcgctcggcccgggtggtctctcccgtgagcgggccggcttcgaggtccgtgacgtgcacccctcgcact

SEQ ID NO: 24 - DNA, 306 bp, S. alboniger
tgcgcaacgtcggcgagctcatccagaaccaggtccgcacgggtctggctcgtatggagcgcgtcgtccgcgagcgcatgacgactcaggacgtcgaggcgatcacgccgcagaccctgatcaacatccggccggtcgtcgcctccatcaaggagttcttcggcaccagccagctgtcgcagttcatggaccagaacaacccgctgtcgggtctcacccacaagcgccgcctgtcggcgctcggaccgggtggtctctcccgtgagcgggccggcttcgaggtccgtgacgtgcacccgtctcact

SEQ ID NO: 25 - DNA, 306 bp, S. albosporeus
tgcgcaacgtcggcgagctcatccagaaccaggtccgcacgggtctggctcgtatggagcgcgtcgtgcgcgagcgcatgacgacccaggacgtcgaggcgatcacgccgcagaccctgatcaacatccgcccggtcgtggcgtcgatcaaggagttcttcggcacctcgcagctgtcgcagttcatggaccagaacaacccgctgtcgggtctgacgcacaagcgtcgtctctcggcgctgggtcccggcggtctgtcccgtgagcgggccggctttgaggtccgtgacgtgcacccgtcgcact

SEQ ID NO: 26 - DNA, 306 bp, S. alboviridis
tgcgcaacgtcggcgagctcatccagaaccaggtccgtacgggcctggcgcggatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcggggctgacccacaagcgccgcctgtccgctcttggcccgggtggtctctcccgtgagcgggccggcttcgaggtccgtgacgtgcacccgtcccact

SEQ ID NO: 27 - DNA, 306 bp, S. albulus
tgcgcaacgtcggcgagctgatccagaaccaggtccgtacgggtctcgcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaccaacccgctgtcgggtctgacccacaagcgtcgtctgtcggcgctgggcccgggtggtctctcccgtgagcgggccggtctggacgtccgtgacgtgcacccgtcgcact

SEQ ID NO: 28 - DNA, 306 bp, S. albus
tgcgcaacgtcggcgagctgatccagaaccaggtccgtacgggtctggcgcgtatggagcgggtcgtccgcgagcggatgacgacgcaggacgtcgaggcgatcacgccgcagacgctgatcaacatccggccggtcgtcgcctccatcaaggagttcttcggcaccagccagctgtcgcagttcatggaccagaccaacccgatctcgggtctgacccacaagcgccgtctcaacgcgctgggcccgggtggtctgagccgtgagcgggccggcttcgaggtccgtgacgtgcacccctcgcact

SEQ ID NO: 29 - DNA, 306 bp, S. almquistii
tgcgcaacgtcggcgagctgatccagaaccaggtccgtacgggtctggcgcgtatggagcgggtcgtccgcgagcggatgacgacgcaggacgtcgaggcgatcacgccgcagacgctgatcaacatccggccggtcgtcgcctccatcaaggagttcttcggcaccagccagctgtcgcagttcatggaccagaccaacccgatctcgggtctgacccacaagcgccgtctgtcggcgctgggcccgggtggtctgagccgtgagcgggccggcttcgaggtccgtgacgtgcacccgtcgcact

SEQ ID NO: 30 - DNA, 306 bp, S. aminophilus
tgcgcaacgtcggcgagctgatccagaaccaggtccgtacgggtctcgcccgtatggagcgcgtcgtgcgcgagcgcatgaccacgcaggacgtcgaggcgatcacgccgcagacgctgatcaacatccggccggtcgtcgcctccatcaaggagttcttcggcaccagccagctgtcgcagttcatggaccagacgaacccgatctcgggtctcacccacaagcggcgtctgaactcgctgggcccgggtggtctgagccgtgagcgggccggcttcgaggtccgtgacgtgcacccctcgcact

SEQ ID NO: 31 - DNA, 306 bp, S. antimycoticus
tgcgcaacgtcggcgagctcatccagaaccaggtccgcacgggtctggctcgtatggagcgcgtcgtgcgtgagcgcatgacgacgcaggacgtcgaggcgatcacgccgcagacgctgatcaacatccggccggtcgtcgcctccatcaaggagttcttcggcaccagccagctgtcgcagttcatggaccagaacaacccgctgtcgggtctcacccacaagcgccgtctgtcggctcttggcccgggtggtctctcccgtgagcgggccggcttcgaggtccgtgacgtgcacccgtctcact

SEQ ID NO: 32 - DNA, 306 bp, S. argenteolus
tgcgcaacgtcggcgagctgatccagaaccaggtccgtacgggtctcgcccgtatggagcgcgtcgtgcgtgagcgcatgaccacccaggacgtcgaggcgatcacgccgcagaccctgatcaacatccggccggtcgtcgcctccatcaaggagttcttcggcaccagccagctgtcgcagttcatggaccagaccaacccgctgtcgggtctgacccacaagcgccgcctgtcggcgctgggcccgggtggcctctcccgtgagcgggccggcctggacgtccgtgacgtgcacccctcgcact

SEQ ID NO: 33 - DNA, 306 bp, S. armeniacus
tgcggaacgtcggcgagctcatccagaaccaggtccgtacgggcctggcgcggatggagcgcgtcgtccgcgagcggatgacgacgcaggacgtcgaggcgatcacgccgcagacgctgatcaacatccgcccggtcgtcgcctccatcaaggaattcttcggcaccagccagctgtcgcagttcatggaccagacgaacccgctgtcggggctcacccacaagcgccgcctcaacgcgctgggcccgggcggtctgagccgtgagcgggcgggcttcgaggtccgtgacgtgcacccctcgcact

SEQ ID NO: 34 - DNA, 306 bp, S. avidinii
tccgcagcgtcgccgagctgatccagaaccaggtccgtacgggtctggcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcggggctgacgcacaagcgtcgtctgaacgcgctcggcccgggtggtctctcccgtgagcgggccggcttcgaggtccgtgacgtgcacccgtcgcact

SEQ ID NO: 35 - DNA, 306 bp, S. bacillaris
tccgcaacgtcggcgagctcatccagacccaggtccgtacgggtctggcccggatggagcgcgtcgtgcgcgagcgcatgacgacccaggacgtcgaggcgatcacgccgcagaccctgatcaacatccggccggtcgtcgcctccatcaaggagttcttcggcaccagccagctgtcgcagttcatggaccagaacaacccgctctcgggtctcacccacaagcgccgtctctcggcgctgggcccgggtggtctctcgcgtgagcgggccggcttcgaggtccgtgacgtgcacccgtcccact

SEQ ID NO: 36 - DNA, 306 bp, S. bambergiensis
tgcgcaacgtcggcgagctcatccagaaccaggtccgtacgggtctggctcgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcgggtctgacccacaagcgccgtctgtcggcgctgggcccgggtggtctgtcccgtgagcgggccggcttggaggtccgtgacgtgcacccctcgcact

SEQ ID NO: 37 - DNA, 306 bp, S. bikiniensis
tgcgcaacgtcggcgagctgatccagaaccaggtccgtacgggtctcgcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcgcagttcatggaccagaacaacccgctgtcgggtctgacgcacaagcgtcgcctctcggcgctcggtcccggcggtctgtcccgtgagcgggccggcttcgaggtccgagacgtgcacccgtcccact

SEQ ID NO: 38 - DNA, 306 bp, S. cacaoi asoensis
tgcgcaacgtcggcgagctcatccagaaccaggtccgcacgggtctggctcgtatggagcgcgtcgtccgcgagcgcatgacgactcaggacgtcgaggccatcacgccgcagaccctgatcaacatccggccggtcgtcgcctccatcaaggagttcttcggcaccagccagctgtcgcagttcatggaccagaacaacccgctctcgggcctgacccacaagcgccgcctgtcggcgctgggccccggcggtctgtcccgtgagcgggccggcttcgaggtccgtgacgttcacccgtcgcact

SEQ ID NO: 39 - DNA, 306 bp, S. capillispiralis
tccgcaccgtcggcgagctgatccagaaccaggtccgtacgggtctcgcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcgggcctgacgcacaagcgtcgtctcaacgccctcggcccgggtggtctctcccgtgagcgggccggcttcgaggtccgcgacgtgcacccgtcgcact

SEQ ID NO: 40 - DNA, 306 bp, S. carpinensis
tgcgcagcgtcggcgagctcatccagaaccaggtccgcacgggtctggcccgtatggagcgcgtcgtccgcgagcggatgaccacgcaggacgtcgaggcgatcacgccgcagaccctgatcaacatccggccggtcgtcgcctccatcaaggagttcttcggcaccagccagctgtcccagttcatggaccagaacaacccgctctccgggctgacgcacaagcggcgtctgtccgcgctggggccgggtggtccctcccgtgagcgggccggcttcgaggtccgtgacgtgcacccgtcgcact

SEQ ID NO: 41 - DNA, 306 bp, S. catenulae
tgcgcaacgtcggcgagctgatccagaaccaggtccgtacgggtctcgcccgtatggagcgcgtcgtgcgtgagcgcatgaccacccaggacgtcgaggcgatcacgccgcagaccctgatcaacatccggccggtcgtcgcctccatcaaggagttcttcggcaccagccagctgtcgcagttcatggaccagacgaacccgctgtcgggtctgacccacaagcgccgtctgtccgcgctgggccccggcggtctctcccgtgagcgggccggtctggacgtccgtgacgtgcacccctcgcact

SEQ ID NO: 42 - DNA, 306 bp, S. celluloflavus
tgcgcaacgtcggcgagctcatccagaaccaggtccgtacgggtctggcgcggatggagcgcgtcgtgcgcgagcgcatgacgacccaggacgtcgaggcgatcacgccgcagaccctgatcaacatccggccggtcgtcgcctccatcaaggagttcttcggcaccagccagctgtcgcagttcatggaccagaacaacccgctctcgggtctcacccacaagcgccgtctctcggcgctcggcccgggtggtctctcgcgtgagcgggccggcttcgaggtccgtgacgtgcacccgtcccact

SEQ ID NO: 43 - DNA, 306 bp, S. chartreusis
tgcgcagcgtcggcgagctcatccagaaccaggtccgcacgggtctggctcgtatggagcgcgtcgtccgtgagcgcatgacgactcaggacgtcgaggcgatcacgccgcagaccctgatcaacatccggccggtcgtcgcctccatcaaggagttcttcggcaccagccagctgtcgcagttcatggaccagaacaacccgctgtcgggtctcacccacaagcgccgtctgtcggctcttggcccgggtggtctctcccgtgagcgggccggcttcgaggtccgtgacgtgcacccgtctcact

SEQ ID NO: 44 - DNA, 307 bp, S. chattanoogensis
tgcgcacccgtcggcgagctcatccagaaccaggtccgtacgggtctggcccgtatggagcgcgtcgtgcgtgagcgcatgaccacccaggacgtcgaggcgatcacgccgcagaccctgatcaacatccggccggtcgtcgcctccatcaaggagttcttcggcaccagccagctgtcgcagttcatggaccagaccaacccgctgtcgggtctgacccacaagcgccgtctgtcggcgctgggcccgggtggtctctcccgtgagcgggccggcctggacgtccgtgacgtgcacccctcgcact

SEQ ID NO: 45 - DNA, 306 bp, S. chrysomallus
tgcgcaacgtcggcgagctcatccagaaccaggtccgtacgggtctcgcccgcatggagcgcgtcgtgcgcgagcgcatgaccacccaggacgtcgaggcgatcacgccgcagaccctgatcaacatccggccggtcgtcgcctccatcaaggagttcttcggcaccagccagctgtcgcagttcatggaccagacgaacccgctgtcgggcctgacccacaagcgccgtctgtccgccctcggtcccggtggtctctcccgtgagcgcgccggcttcgaggtccgtgacgtgcacccctcgcact

SEQ ID NO: 46 - DNA, 306 bp, S. cinereoruber
tgcgcaacgtcggcgagctcatccagaaccaggtccgtacgggtctggctcgtatggagcgcgtcgtccgcgagcgcatgacgactcaggacgtcgaggccatcacgccgcagaccctgatcaacatccggccggtcgtcgcctccatcaaggagttcttcggcaccagccagctgtcgcagttcatggaccagaacaacccgctgtcgggtctcacccacaagcgccgtctctcggcgctcggtccgggtggtctctcccgtgagcgggccggcttcgaggtccgtgacgtgcacccctcgcact

SEQ ID NO: 47 - DNA, 306 bp, S. cinereus
tccgcaacgtcggcgagctgatccagaaccaggtccgtacgggtctcgcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcggggctgacgcacaagcgtcgtctgaacgccctcggcccgggtggtctctcccgtgagcgggccggcttcgaggtccgcgacgtccacccgtcgcact

SEQ ID NO: 48 - DNA, 306 bp, S. cinnamonensis
tgcgcaacgtcggcgagctcatccagaaccaggtccgcacgggtctggctcgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcgcagttcatggaccagaacaacccgctctcgggcctgacccacaagcgccgcctgtcggcgctgggccccggcggtctgtcccgtgagcgggccggcttcgaggtccgtgacgttcacccgtcgcact

SEQ ID NO: 49 - DNA, 306 bp, S. cirratus
tgcgcaacgtcggcgagctcatccagaaccaggtccgtacgggtctggctcgtatggagcgcgtcgtgcgcgagcgcatgaccacgcaggacgtcgaggcgatcacgccgcagaccctgatcaacatccggccggtcgtcgcctccatcaaggagttcttcggcaccagccagctgtcgcagttcatggaccagaacaacccgctgtcgggcctgacccacaagcgccgtctgtcggcgctgggccccggtggtctgtcccgtgagcgggccggcttcgaggtccgtgacgtccacccgtcgcact

SEQ ID NO: 50 - DNA, 306 bp, S. clavuligerus
tgcgcaacgtcagcgagctgatccagaaccaggtccgtacgggtctcgcccgtatggagcgtgtcgtgcgcgagcgcatgaccacccaggacgtcgaggcgatcacgccccagaccctgatcaacatccggccggtcgtcgcctccatcaaggagttcttcggcaccagccagctgtcgcagttcatggaccagaacaacccgctgtcgggtctcacccacaagcgccgtctgtcggcgctcggcccgggtggtctctcccgtgagcgggcgggcttcgaggtccgtgacgtgcacccctcgcact

SEQ ID NO: 51 - DNA, 306 bp, S. coelicolor
tccgcaacgtcggcgagctgatccagaaccaggtccgtacgggtctcgcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcggggctgacgcacaagcgtcgtctgaacgccctcggcccgggtggtctctcccgtgagcgggccggcttcgaggtccgcgacgtgcacccgtcgcact

SEQ ID NO: 52 - DNA, 306 bp, S. coeruleorubidus
tgcgcagcgtcggcgagctcatccagaaccaggtccgtacgggtctggcgcgtatggagcgtgtcgtgcgcgagcggatgacgacccaggacgtcgaggcgatcacgccgcagaccctgatcaacatccggccggtcgtcgcctccatcaaggagttcttcggcaccagccagctgtcccagttcatggaccagaacaacccgctgtccggtctgacgcacaagcggcgtctgtccgcgctcggcccgggtggtctgtcccgtgagcgggccggcttcgaggtccgtgacgtgcacccctcgcact

SEQ ID NO: 53 - DNA, 306 bp, S. collinus
tgcgcagcgtcggcgagctcatccagaaccaggtccgcacgggtctggcgcgtatggagcgtgtcgtccgcgagcggatgacgacccaggacgtcgaggcgatcacgccgcagaccctgatcaacatccggccggtcgtcgcctccatcaaggagttcttcggcaccagccagctgtcgcagttcatggaccagaacaacccgctgtcgggtctcacccacaagcgccgtctgtcggctcttggcccgggtggtctgtcccgtgagcgggccggcttcgaggtccgtgacgtgcacccctcgcact

SEQ ID NO: 54 - DNA, 306 bp, S. corchorusii
tgcgcagcgtcggcgagctcatccagaaccaggtccgcacgggtctggcgcgtatggagcgtgtcgtccgcgagcggatgacgacccaggacgtcgaggcgatcacgccgcagaccctgatcaacatccggccggtcgtcgcctccatcaaggagttcttcggcaccagtcagctgtcccagttcatggaccagaacaacccgctgtcgggtctcacccacaagcgccgtctgtcggctcttggcccgggtggtctctcccgtgagcgggccggcttcgaggtccgtgacgtgcacccgtcgcact

SEQ ID NO: 55 - DNA, 306 bp, S. crystallinus
tgcgcaacgtcggcgagctcatccagaaccaggtccgtacgggtctggcgcgtatggagcgggtcgtccgcgagcggatgacgactcaggacgtcgaggcgatcacgccgcagaccctgatcaacatccggccggtcgtcgcctccatcaaggagttcttcggcaccagccagctgtcgcagttcatggaccagaacaacccgctgtcgggtctcacccacaagcgccgtctgtcggcgcttggcccgggtggtctctcccgtgagcgggccggcttcgaggtccgtgacgtgcacccgtctcact

SEQ ID NO: 56 - DNA, 306 bp, S. cuspidosporus
tccgcaacgtcggcgagctgatccagaaccaggtccgcacgggtctggcccgtatggagcgcgtcgtccgcgagcgcatgacgactcaggacgtcgaggcgatcacgccgcagaccctgatcaacatccggccggtcgtcgcctccatcaaggagttcttcggcaccagccagctgtcgcagttcatggaccagacgaacccgctgtcgggtctgacccacaagcgccgtctgaacgcgctcggccccggtggtctctcccgtgagcgggccggcttcgaggtccgagacgtgcacccgtcgcact

SEQ ID NO: 57 - DNA, 306 bp, S. cyaneus
tgcgcagcgtcggcgagctcatccagaaccaggtccgtaccggtctggctcgtatggagcgcgtcgtccgtgagcgcatgacgacccaggacgtcgaggcgatcacgccgcagaccctgatcaacatccggccggtcgtcgcctccatcaaggagttcttcggcaccagccagctgtcgcagttcatggaccagaacaacccgctgtcgggtctcacccacaagcgccgcctgtcggctcttggcccgggtggtctctcccgtgagcgggccggcttcgaggtccgtgacgtgcacccgtctcact

SEQ ID NO: 58 - DNA, 306 bp, S. diasticus
tgcgtaacgtcggcgagctcatccagaaccaggtccgtacgggtctggctcgtatggagcgcgtcgtgcgtgagcgcatgacgacgcaggacgtcgaggcgatcacgccgcagacgctgatcaacatccggccggtcgtcgcctccatcaaggagttcttcggcaccagccagctgtcgcagttcatggaccagaacaacccgctgtcgggtctcacccacaagcgccgcctgtcggctcttggcccgggtggtctgtcccgtgagcgggccggcttcgaggtccgtgacgtgcacccgtcgcact

SEQ ID NO: 59 - DNA, 306 bp, S. djakartensis
tgcgcagcgtcggcgagctcatccagaaccaggtccgcacgggtctggcgcgtatggagcgtgtcgtccgcgagcggatgacgacccaggacgtcgaggcgatcacgccgcagaccctgatcaacatccggccggtcgtcgcctccatcaaggagttcttcggcaccagccagctgtcccagttcatggaccagaacaacccgctgtcgggtctgacgcacaagcggcgtctgtccgcgctcggcccgggtggtctgtcccgtgagcgggccggcttcgaggtccgtgacgtgcacccgtcgcact

SEQ ID NO: 60 - DNA, 306 bp, S. durhamensis
tccgcagcgtcggtgagctgatccagaaccaggtccgtacgggtctcgcccgtatggagcgtgtcgtgcgcgagcgcatgaccacgcaggacgtcgaggcgatcacgccgcagaccctgatcaacatccgcccggtcgtggcgtcgatcaaggagttcttcggcacctcgcagctgtcccagttcatggaccagaacaacccgctgtcggggctgacgcacaagcgtcgtctgaacgccctcggcccgggtggtctctcccgtgagcgggccggcttcgaggtccgtgacgtgcacccctcgcact

SEQ ID NO: 61 - DNA, 306 bp, S. echinoruber
tgcgcagcgtcggcgagctgatccagaaccaggtgcgcaccggtctcgcccgtatggagcgggtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcggggctgacgcacaagcgtcgtctcaacgccctcggcccgggtggtctctcccgtgagcgggccggcttcgaggtccgcgacgtgcacccgtcgcact

SEQ ID NO: 62 - DNA, 306 bp, S. ederensis
tgcgcaacgtcggcgagctcatccagaaccaggtccgtacgggtctggcgcgtatggagcgtgtcgtccgtgagcggatgacgactcaggacgtcgaggcgatcacgccgcagaccctgatcaacatccggccggtcgtcgcctccatcaaggagttcttcggcaccagccagctgtcgcagttcatggaccagaacaacccgctgtcggggctgacgcacaagcgtcgtctcaacgcgctcggcccgggtggtctctcccgtgagcgggccggcttcgaagtccgtgacgtgcacccctcgcact

SEQ ID NO: 63 - DNA, 306 bp, S. ehimensis
tgcgcaacgtcggcgagctcatccagaaccaggtccgcacgggtctggctcgtatggagcgcgtcgtccgcgagcggatgaccacccaggacgtcgaggcgatcacgccgcagaccctgatcaacatccggccggtcgtcgcctccatcaaggagttcttcggcaccagccagctgtcccagttcatggaccagaccaacccgctgtcgggtctgacccacaagcgtcgtctgtcggcgctgggtccgggcggtctctcccgtgagcgggccggcttcgaggtccgagacgtccacccgtcgcact

SEQ ID NO: 64 - DNA, 306 bp, S. flaveolus
tccgcaacgtcggcgagctgatccagaaccaggtccgtacgggtctcgcccgtatggagcgggtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcgggtctgacgcacaagcgtcgtctcaacgcgctcggcccgggtggtctctcccgtgagcgggccggcttcgaggtccgtgacgttcacccgtcgcact

SEQ ID NO: 65 - DNA, 306 bp, S. flavofuscus
tgcgcagcgtcggcgagctgatccagaaccaggtccgcacgggtctggcccgtatggagcgcgtcgtgcgtgagcgcatgacgactcaggacgtcgaggcgatcacgccgcagaccctgatcaacatccggccggtcgtcgcctccatcaaggagttcttcggcaccagccagctgtcgcagttcatggaccagacgaacccgctgtcgggtctgacccacaagcgccgtctgaacgcgctcggtcccggtggtctgtcccgcgagcgggcgggtttcgaggtccgtgacgtgcacccgtcgcact

SEQ ID NO: 66 - DNA, 306 bp, S. fradiae
tgcgcaacgtcggcgagctcatccagaaccaggtccgcacgggtctggcgcggatggagcgcgtcgtccgcgagcgcatgaccacccaggacgtcgaggcgatcacgccgcagaccctgatcaacatccggccggtcgtcgcctccatcaaggagttcttcggcaccagccagctgtcgcagttcatggaccagaacaacccgctgtccgggctgacgcacaagcgtcgtctgtccgcgctcggcccgggtggtctctcccgtgagcgggccggcttcgaggtccgagacgtgcacccgtcccact

SEQ ID NO: 67 - DNA, 306 bp, S. galilaeus
tccgcaacgtcggcgagctgatccagaaccaggtccgtacgggtctcgcccgtatggagcgggtcgtgcgtgagcgcatgaccactcaggacgtcgaggcgatcacgccgcagaccctgatcaacatccggccggtcgtcgcctccatcaaggagttcttcggcaccagccagctgtcccagttcatggaccagaacaacccgctgtcggggctgacgcacaagcgtcgtctcaacgccctcggcccgggtggtctgtcccgtgagcgggccggcttcgaggtccgtgacgttcacccgtcgcact

SEQ ID NO: 68 - DNA, 306 bp, S. globisporus
tgcgcaacgtgggcgagctcatccagaaccaggtccgtacgggcctggcgcggatggagcgcgtcgtgcgcgagcgcatgaccacccaggacgtcgaggcgatcacgccgcagaccctgatcaacatccggccggtcgtcgcctccatcaaggagttcttcggcaccagccagctgtcgcagttcatggaccagaacaacccgctgtcgggtctcacccacaagcgccgtctgtcggctcttggcccgggtggtctctcccgtgagcgggccggcttcgaggtccgagacgtgcacccgtcccact

SEQ ID NO: 69 - DNA, 306 bp, S. griseochromogenes
tgcgcagcgtcggcgagctgatccagaaccaggtccgtacgggtctcgcccgtatggagcgtgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcgggtctcacccacaagcgccgtctgtcggcgctcggcccgggtggtctctcccgtgagcgggccggcttcgaggtccgtgacgtgcacccgtcgcact

SEQ ID NO: 70 - DNA, 306 bp, S. griseolus
tgcgcaacgtcggcgagctcatccagaaccaggtccgtacgggtctcgcccgtatggagcgggtcgtccgcgagcgcatgaccacccaggacgtcgaggcgatcacgccgcagaccctgatcaacatccggccggtcgtcgcctccatcaaggagttcttcggcaccagccagctgtcgcagttcatggaccagaacaacccgctgtccggtctcacccacaagcgccgtctctcggcgctcggcccgggtggtctctcgcgtgagcgggccggcttcgaggtccgtgacgtgcacccgtcccact

SEQ ID NO: 71 - DNA, 306 bp, S. griseoviridis
tgcgcagcgtcggcgagctcatccagaaccaggtccgtacgggtctggcgcgtatggagcgcgtcgtgcgcgagcgcatgacgacccaggacgtcgaggcgatcacgccgcagacgctgatcaacattcggccggtcgtcgcctccatcaaggagttcttcggcaccagccagctgtcgcagttcatggaccagaacaacccgctgtcgggtctcacccacaagcgccgcctgtcggcgctcggtccgggtggtctctcccgtgagcgggccggcttcgaggtccgtgacgtgcacccctcgcact

SEQ ID NO: 72 - DNA, 306 bp, S. griseus griseus
tgcgcaacgtcggcgagctgatccagaaccaggtccgtacgggtctggcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcggggctgacgcacaagcgtcgtctgaacgctctcggcccgggtggtctctcccgtgagcgggccggcttcgaggtccgagacgtgcacccgtcgcact

SEQ ID NO: 73 - DNA, 306 bp, S. hiroshimensis
tgcgcaacgtcggcgagctcatccagaaccaggtccgcacgggtctggctcgtatggagcgcgtcgtccgcgagcggatgaccacccaggacgtcgaggcgatcacgccgcagaccctgatcaacatccggccggtcgtcgcctccatcaaggagttcttcggcaccagccagctgtcccagttcatggaccagaccaacccgctgtcgggtctgacccacaagcgccgtctctcggcgctgggcccgggtggtctctcccgtgagcgggccggcttcgaggtccgtgacgttcacccgtcgcact

SEQ ID NO: 74 - DNA, 306 bp, S. hygroscopicus
tgcgcaacgtcggcgagctcatccagaaccaggtccgcacgggtctggctcgtatggagcgcgtcgtgcgtgagcgcatgacgacccaggacgtcgaggcgatcacgccgcagaccctgatcaacatccggccggtcgtcgcctccatcaaggagttcttcggcaccagccagctgtcccagttcatggaccagaccaacccgctgtcgggtctgacccacaagcgccgtctgtcggcgctgggcccgggtggtctctcccgtgagcgggccggcctggacgtccgtgacgtgcacccctcgcact

SEQ ID NO: 75 - DNA, 306 bp, S. libani libani
tccgcaacgtcggcgagctgatccagacccaggtccgtacgggtctggctcggatggagcgcgtcgtgcgtgagcgcatgaccacccaggacgtcgaggcgatcacgccgcagaccctgatcaacatccggccggtcgtcgcctccatcaaggagttcttcggcaccagcaagttgtccaagttcatggaccagaccaacccgctgtcgggtctgacccacaagcgtcgtctgtccgcgctgggcccgggtggtctctcccgtgagcgggccggcttcgacgtccgtgacgtgcacccctcgcact

SEQ ID NO: 76 - DNA, 306 bp, S. limosus
tgcgcaacgtcggcgagctgatccagaaccaggtccgcacgggtctcgcccgtatggagcgtgtcgtgcgtgagcgcatgaccacccaggacgtcgaggcgatcacgccgcagaccctgatcaacatccggccggtcgtcgcctccatcaaggagttcttcggcaccagccagctgtcgcagttcatggaccagaacaacccgctctcgggtctcacccacaagcgccgtctgtcggcgctcggcccgggtggtctctcgcgtgagcgggccggtttcgaggtccgtgacgtgcacccctcgcact

SEQ ID NO: 77 - DNA, 306 bp, S. lincolnensis
tgcgcagcgtcggcgagctcatccagaaccaggtccgtacgggtctggcgcgtatggagcgagtcgtccgtgagcggatgacgacccaggacgtcgaggcgatcacgccgcagaccctgatcaacatccggccggtcgtcgcctccatcaaggagttcttcggcaccagccagctctcgcagttcatggaccagaacaacccgctgtccggtctgacgcacaagcgtcgtctctcggcgctgggccccggtggtctgtcgcgtgagcgggccggcttcgaggtccgtgacgtgcacccgccccact

SEQ ID NO: 78 - DNA, 306 bp, S. longwoodensis
tgcgcaacgtcggcgagctcatccagaaccaggtccgtacgggcctggcgcgtatggagcgcgtcgtccgcaagcgcatgacgacccaggacgtcgaggcgatcacgccgcagaccctgatcaacatccggcccgtcgtcgcctccatcaaggagttcttcggcaccagccagctgtcgcagttcatggaccagaacaacccgctgtcgggtctcacccacaagcgccgtctgtcggctcttggcccgggtggtctctcccgtgagcgggccggcttcgaggtccgtgacgtgcacccctcgcact

SEQ ID NO: 79 - DNA, 306 bp, S. melanogenes
tgcgcaacgtcggcgagctcatccagaaccaggtccgtacgggtctggcgcgtatggagcgcgtcgtgcgtgagcgcatgacgacccaggacgtcgaggcgatcacgccgcagaccctgatcaacatccggccggtcgtcgcctccatcaaggagttcttcggcaccagccagctgtcgcagttcatggaccagaacaacccgctgtcgggtctcacccacaagcgccgtctgtcggcgcttggcccgggtggtctctcccgtgagcgggccggcttcgaggtccgtgacgtgcacccgtctcact

SEQ ID NO: 80 - DNA, 306 bp, S. minutiscleroticus
tgcgcaacgtcggcgagctcatccagaaccaggtccgcacgggtctggcccgtatggagcgcgtcgtgcgcgagcgcatgaccacccaggacgtcgaggcgatcacgccgcagaccctgatcaacatccggccggtcgtcgcctccatcaaggagttcttcggcaccagccagctctcgcagttcatggaccagaacaacccgctgtcgggtctgacgcacaagcgtcgcctctcggcgctgggtcccggcggtctgtcgcgtgagcgggccggcttcgaggtccgtgacgtgcacccctcgcact

SEQ ID NO: 81 - DNA, 306 bp, S. nitrosporeus
tgcgtaacgtcggcgagctcatccagaaccaggtccgcacgggtctcgcccgtatggagcgcgtcgtgcgcgagcgcatgacgacccaggacgtcgaggcgatcacgccgcagaccctgatcaacatccggccggtcgtcgcctccatcaaggagttcttcggcaccagccagctgtcgcagttcatggaccagaacaacccgctctcgggtctcacccacaagcgccgtctctcggcgctcggcccgggtggtctctcgcgtgagcgggccggcttcgaggtccgcgacgttcacccgtcccact

SEQ ID NO: 82 - DNA, 306 bp, S. noboritoensis
tgcgcaacgtcggcgagctcatccagaaccaggtccgtacgggtctggcgcgtatggagcgcgtcgtgcgtgagcgcatgacgacccaggacgtcgaggcgatcacgccgcagaccctgatcaacatccggccggtcgtcgcctccatcaaggagttcttcggcaccagccagctgtcgcagttcatggaccagaacaacccgctgtgcggtctcacccacaagcgccgtctgtcggcgctgggcccgggtggtctctcccgtgagcgggccggcttcgaggtccgtgacgtgcacccgtctcact

SEQ ID NO: 83 - DNA, 306 bp, S. nodosus
tgcgcagcgtcggcgagctcatccagaaccaggtccgcacgggtctggcgcgtatggagcgcgtcgtgcgcgagcgcatgaccacgcaggacgtcgaggcgatcacgccgcagaccctgatcaacatccggccggtcgtcgcctccatcaaggagttcttcggcaccagccagctgtcccagttcatggaccagaacaacccgctgtccggcctgacgcacaagcggcgtctgtccgcgctgggcccgggcggtctctcccgtgagcgggccggcttcgaggtccgtgacgtgcacccctcgcact

SEQ ID NO: 84 - DNA, 306 bp, S. nojiriensis
tgcgcaacgtcggcgagctcatccagaaccaggtccgcacgggtctggctcgtatggagcgcgtcgtccgcgagcgcatgacgacccaggacgtcgaggccatcacgccgcagaccctgatcaacatccggccggtcgtcgcctccatcaaggagttcttcggcaccagccagctgtcgcagttcatggaccagaacaacccgctctcgggcctgacccacaagcgccgcctgtcggcgctgggccccggcggtctgtcccgtgagcgggccggcttcgaggtccgtgacgtccacccgtcgcact

SEQ ID NO: 85 - DNA, 306 bp, S. olivaceoviridis
tgcgcagcgtcggcgagctcatccagaaccaggtccgcacgggtctggcgcgtatggagcgagtcgtccgcgagcgcatgaccacccaggacgtcgaggcgatcacgccgcagactctgatcaacatccggccggtcgtcgcctccatcaaggagttcttcggcaccagccagctgtcccagttcatggaccagaacaacccgctgtcgggtctcacccacaagcgccgtctgtcggctcttggcccgggtggtctctcccgtgagcgggccggcttcgaggtccgtgacgtgcacccgtcgcact

SEQ ID NO: 86 - DNA, 306 bp, S. olivochromogenes
tgcgcaacgtcggcgagctcatccagaaccaggtccgcacgggtctggctcgtatggagcgcgtcgtccgcgagcgcatgacgacccaggacgtcgaggcgatcacgccgcagaccctgatcaacatccggcccgtcgtcgcctccatcaaggagttcttcggcaccagccagctgtcgcagttcatggaccagaacaacccgctgtcgggtctcacccacaagcgccgtctgtcggctcttggcccgggtggtctctcccgtgagcgggccggcttcgaggtccgtgacgtgcacccctcgcact

SEQ ID NO: 87 - DNA, 306 bp, S. pactum
tgcgcaacgtcggcgagctcatccagaaccaggtccgcaccggtctggcccgtatggagcgcgtcgtccgcgagcggatgaccacccaggacgtcgaggcgatcacgccgcagaccctgatcaacatccggccggtcgtcgcctccatcaaggagttcttcggcaccagccagctgtcgcagttcatggaccagaccaacccgctgtcgggtctggcccacaagcgccgcctgtcggcgctgggcccgggtggtctgtcccgtgagcgggccggcttcgaggtccgtgacgtgcacccgtcgcact

SEQ ID NO: 88 - DNA, 306 bp, S. paradoxus
tgcgcagcgtcggcgagctcatccagaaccaggtccgtacgggcctggcgcgtatggagcgtgtcgtccgcgagcggatgacgacccaggacgtcgaggcgatcacgccgcagaccctgatcaacatccggccggtcgtcgcctccatcaaggagttcttcggcaccagccagctgtcgcagttcatggaccagaacaacccgctgtcgggtctcacccacaagcgccgtctgtcggcgcttggcccgggtggtctctcccgtgagcgggccggcttcgaggtccgtgacgtgcacccctcgcact

SEQ ID NO: 89 - DNA, 306 bp, S. peucetius
tgcgcaacgtcggcgagctcatccagaaccaggtccgcacgggtctggctcgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcgcagttcatggaccagaacaacccgctgtcgggtctcacccacaagcgccgtctgtcggctcttggtccgggtggtctctcccgtgagcgggccggcttcgaggtccgtgacgtgcacccgtcgcact

SEQ ID NO: 90 - DNA, 306 bp, S. phaeochromogenes
tgcgcaacgtcggcgagctcatccagaaccaggtccgtacgggtctggcgcgtatggagcgtgtcgtccgtgagcggatgacgactcaggacgtcgaggcgatcacgccgcagaccctgatcaacatccggccggtcgtcgcctccatcaaggagttcttcggcaccagccagctgtcgcagttcatggaccagaacaacccgctgtcgggcctgacgcacaagcgtcgtctcaacgcgctcggcccgggtggtctgtcccgtgagcgggccggcttcgaagtccgtgacgtgcacccctcgcact

SEQ ID NO: 91 - DNA, 306 bp, S. plicatus
tccgcagcgtcggcgagctgatccagaaccaggtccgtacgggtctcgcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcgggtctgacgcacaagcgtcgtctcaacgcgctcggcccgggtggtctctcccgtgagcgggccggcttcgaggtccgtgacgtccacccctcgcact

SEQ ID NO: 92 - DNA, 306 bp, S. pulveraceus
tccgtaacgtcggcgagctgatccagaaccaggtccgtacgggtctcgcccgtatggagcgtgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtccggcctgacgcacaagcgccgtctcaacgcgctgggccccggtggtctctcccgtgagcgggccggcttcgaagtccgtgacgtgcacccgtcgcact

SEQ ID NO: 93 - DNA, 306 bp, S. rameus
tgcgcagcgtcggcgagctcatccagaaccaggtccgtacgggtctggcgcgtatggagcgtgtcgtccgcgagcgcatgacgacccaggacgtcgaggcgatcacgccgcagaccctgatcaacatccggccggtcgtcgcctccatcaaggagttcttcggcaccagccagctgtcccagttcatggaccagaacaacccgctgtcgggtctcacccacaagcgccgtctgtcggctcttggcccgggtggtctctcccgtgagcgggccggcttcgaggtccgtgacgtgcacccgtcgcact

SEQ ID NO: 94 - DNA, 306 bp, S. rimosus rimosus
tccgcaacgtcggcgagctgatccagaaccaggtccgtacggctctcgcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaccaacccgctgtcgggcctgacccacaagcgccgtctgtcggcgctgggccccggtggtctctcccgtgagcgggccggcctggacgtccgtgacgtgcacccctcgcact

SEQ ID NO: 95 - DNA, 306 bp, S. roseosporus
tgcgcagcgtcggcgagctcatccagaaccaggtccgtacgggtctggcgcgtatggagcgtgtcgtccgcgagcggatgacgacccaggacgtcgaggcgatcacgccgcagaccctgatcaacatccggccggtcgtcgcctccatcaaggagttcttcggcaccagccagctgtcccagttcatggaccagaacaacccgctgtcgggtctgacgcacaagcggcgtctgtccgcgctcggcccgggtggtctgtcccgtgagcgggccggcttcgaggtccgtgacgtgcacccctcgcact

SEQ ID NO: 96 - DNA, 306 bp, S. sclerotialus
tccgtaacgtcggtgagctgatccagaaccaggtccgtacgggtctcgcccgtatggagcgcgtcgtgcgcgagcgcatgaccacccaggacgtcgaggcgatcacgccgcagaccctgatcaacatccggccggtcgtcgcccccatcaaggagttcttcggcaccagccagctgtcccagttcatggaccagaacaacccgctgtcgggcctgacgcacaagcgtcgtctcaacgccctcggcccgggtggtctctcccgtgagcgggccggcttcgaggtccgtgacgttcacccctcgcact

SEQ ID NO: 97 - DNA, 306 bp, S. setonii
tgcgcagcgtcggcgagctcatccagaaccaggtccgcacgggtctggcgcggatggagcgcgtcgtgcgcgagcgcatgaccacgcaggacgtcgaggcgatcacgccgcagaccctgatcaacatccggccggtcgtcgcctccatcaaggagttcttcggcaccagccagctgtcccagttcatggaccagaacaacccgctgtccggtctgacgcacaagcgtcgtctgtccgcgctcggcccgggtggtctctcccgtgagcgggccggcttcgaggtccgtgacgtgcacccctcgcact

SEQ ID NO: 98 - DNA, 306 bp, S. sioyaensis
tgcgcaacgtcggcgagctcatccagaaccaggtccgtacgggtctggcgcgtatggagcgcgtcgtgcgtgagcgcatgacgacccaggacgtcgaggcgatcacgccgcagaccctgatcaacatccggccggtcgtcgcctccatcaaggagttcttcggcaccagccagctgtcccagttcatggaccagaccaacccgctgtcgggtctgacccacaagcgccgtctgtcggcgctgggcccgggtggtctctcccgtgagcgggccggtctggacgtccgtgacgtgcacccctcgcact

SEQ ID NO: 99 - DNA, 306 bp, S. somaliensis
tgcgcaccgtcggcgagctcatccagacccaggtccgcacgggcctggcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcgcagttcatggaccagaacaacccgctgtccggtctgacgcacaagcgccgtctgtccgcgctgggcccgggtggtctctcccgtgagcgggccggcttcgaggtccgtgacgtgcacccgtcccact

SEQ ID NO: 100 - DNA, 306 bp, S. spectabilis
tgcgcaacgtcggcgagctgatccagaaccaggtccgtacgggtctcgcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcgcagttcatggaccagaacaacccgctgtcgggcatcacccacaagcggcgtctgaactcgctcggcccgggtggtctctcccgtgagcgggcgggcttcgaggtccgtgacgtgcacccctcgcact

SEQ ID NO: 101 - DNA, 306 bp, S. subrutilus
tgcgcaacgtcggcgagctcatccagaaccaggtccgcacgggtctggctcgtatggagcgcgtcatccgcgagcggatgacgacccaggacgtcgaggcgatcacgccgcagaccctgatcaacatccggccggtcgtcgcctccatcaaggagttcttcggcaccagccagctgtcgcagttcatggaccagaacaacccgctgtcgggtctgacgcacaagcgtcgtctctcggctctgggacccggcggtctgtcccgtgagcgggccggcttcgaggtccgtgacgtgcacccgtcgcact

SEQ ID NO: 102 - DNA, 306 bp, S. tubercidicus
tgcgcaacgtcggcgagctcatccagaaccaggtccgtacgggtctggcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcgcagttcatggaccagaccaacccgctgtcgggtctgacccacaagcgtcgtctgtcggcgctgggcccgggtggtctgtcccgtgagcgggccggcttcgaggtccgtgacgtgcacccctcgcact

SEQ ID NO: 103 - DNA, 306 bp, S. vinaceus
tgcgcaacgtcggcgagctcatccagaaccaggtccgcacgggtctggcccgtatggagcgcgtcgtccgcgagcgcatgaccacgcaggacgtcgaggcgatcacgccgcagaccctgatcaacatccggccggtcgtcgcctccatcaaggagttcttcggcaccagccagctgtcgcagttcatggaccagaacaacccgctgtcgggtctgacgcacaagcgtcgtctttcggcgctgggtcccggtggtctgtcccgtgagcgggccggcttcgaggtccgtgacgtgcacccgtcgcact

SEQ ID NO: 104 - DNA, 306 bp, S. violarus
tgcgcagcgtcggcgagctcatccagaaccaggtccgtacgggtctggcgcgtatggagcgtgtcgtccgcgagcggatgacgacccaggacgtcgaggcgatcacgccgcagaccctgatcaacatccggccggtcgtcgcctccatcaaggagttcttcggcaccagccagctgtcgcagttcatggaccagaacaacccgctgtcgggcctcacccacaagcgccgtctgtcggctcttggcccgggtggtctgtcccgtgagcgggccggcttcgaggtccgtgacgtgcacccctcgcact

SEQ ID NO: 105 - DNA, 306 bp, S. violascens
tgcgcaacgtcggcgagctcatccagaaccaggtccgtacgggtctggctcgtatggagcgcgtcgtgcgtgagcgcatgacgacccaggacgtcgaggcgatcacgccgcagaccctgatcaacatccggccggtcgtcgcctccatcaaggagttcttcggcaccagccagctgtcgcagttcatggaccagaacaacccgctgtcgggtctcacccacaagcgccgtctgtcggcgctcggcccgggtggtctctcgcgtgagcgggccggcttcgaggtccgtgacgtgcacccgtctcact

SEQ ID NO: 106 - DNA, 306 bp, S. virginiae
tgcgcaacgtcggcgagctcatccagaaccaggtccgcacgggtctggctcgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcgcagttcatggaccagaacaacccgctctcgggcctgacccacaagcgccgtctgtcggcgctgggccccggcggtctgtcccgtgagcgggccggcttcgaggtccgtgacgtccacccgtcgcact

SEQ ID NO: 107 - DNA, 306 bp, S. xantophaeus
tgcgcaacgtcggcgagctcatccagaaccaggtccgcacgggtctggctcgtatggagcgcgtcgtccgcgagcgcatgacgactcaggacgtcgaggccatcacgccgcagaccctgatcaacatccggccggtcgtcgcctccatcaaggagttcttcggcaccagccagctgtcgcagttcatggaccagaacaacccgctgtcgggcctcacccacaagcgccgcctgtcggctcttggtcccggtggtctgtcccgtgagcgggccggcttcgaggtccgtgacgtgcacccgtcgcact

SEQ ID NO: 108 - DNA, 306 bp, S.albaduncus
tgcgcaacgtcggcgagctgatccagaaccaggtccgtacgggtctcgcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcggggctgacgcacaagcgtcgtctgaacgccctcggcccgggtggtctctcccgtgagcgggccggcttcgaggtccgcgacgtccacccgtcgcact

SEQ ID NO: 109 - DNA, 306 bp, S.althioticus
tccgcaacgtcggcgagctgatccagaaccaggtccgtacgggtctcgcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcggggctgacgcacaagcgtcgtctgaacgccatcggcccgggtggtctctcccgtgagcgggccggcttcgaggtccgtgacgtgcacccctcgcact

SEQ ID NO: 110 - DNA, 306 bp, S.ambofaciens
tccgcaacgtcggcgagctgatccagaaccaggtccgtacgggtctcgcccgtatggagcgggtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcggggctgacgcacaagcgtcgtctgaacgccctcggcccgggtggtctctcccgtgagcgggccggcttcgaggtccgcgacgtccacccgtcgcact

SEQ ID NO: 111 - DNA, 306 bp, S.anulatus
tgcgcaacgtcggcgagctcatccagaaccaggtccgtacgggtctggcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcgcagttcatggaccagaacaacccgctgtcgggcctgacccacaagcgccgtctgaacgccctgggcccgggtggtctgtcccgtgagcgggccggcttcgaggtccgtgacgtgcacccgtcgcact

SEQ ID NO: 112 - DNA, 306 bp, S.anthocyanicus; identical to SEQ ID NO: 51

SEQ ID NO: 113 - DNA, 306 bp, S.cellulose; identical to SEQ ID NO: 22

SEQ ID NO: 114 - DNA, 306 bp, S.armeniacus; identical to SEQ ID NO: 22

SEQ ID NO: 115 - DNA, 306 bp, S.coelescens
tgcgcaacgtcggcgagctgatccagaaccaggtccgtacgggtctcgcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcggggctgacgcacaagcgtcgtctgaacgccctcggcccgggtggtctctcccgtgagcgggccggcttcgaggtccgcgacgtgcacccgtcgcact

SEQ ID NO: 116 - DNA, 306 bp, S.griseoflavus
tgcgcaacgtcggcgagctgatccagaaccaggtccgcacgggtctcgcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcgggcctgacgcacaagcgtcgtctgaacgcgctcggcccgggtggtctctcccgtgagcgggccggcttcgaggtccgtgacgtgcacccctcgcact

SEQ ID NO: 117 - DNA, 306 bp, S.humiferus; identical to SEQ ID NO: 51

SEQ ID NO: 118 - DNA, 306 bp, S.lividans; identical to SEQ ID NO: 108

SEQ ID NO: 119 - DNA, 306 bp, S.murinus
tccgcaacgtcggcgagctgatccagaaccaggtccgtacgggtctcgcccgtatggagcgcgtcgtgcgcgagcgcatggccacccaggacgtcgaggcgatcacgccgcagaccctgatcaacatccggccggtcgtcgcctccatcaaggagttcttcggcaccagccagctgtcccagtccatggaccagaacaacccgctgtcggggctgacgcacaagcgtcgtctgaacgccctcggcccgggtggtctctcccgtgagcgggccggcttcgaggtccgcgacgtgcacccgtcgcact

SEQ ID NO: 120 - DNA, 306 bp, S.pilosus
tgcgcaacgtcggcgagctgatccagaaccaggtccgtacgggtctcgcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcgggtctgacgcacaagcgtcgtctgaacgcccttggcccgggtggtctctcccgtgagcgggccggctttgaggtccgtgacgtgcacccctcgcact

SEQ ID NO: 121 - DNA, 306 bp, S.rubiginosus
tccgcaacgtcggcgagctgatccagaaccaggtccgtacgggtctcgcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcggggctgacgcacaagcgtcgtctgaacgccctcggcccgggtggcctctcccgtgagcgggccggcttcgaggtccgtgacgtgcacccctcgcact

SEQ ID NO: 122 - DNA, 306 bp, S.tendae; identical to SEQ ID NO: 51

SEQ ID NO: 123 - DNA, 306 bp, S.umbrinus
tgcgcaacgtcggcgagctcatccagaaccaggtccgtacgggtctggcgcgtatggagcgtgtcgtccgtgagcggatgacgactcaggacgtcgaggcgatcacgccgcagaccctgatcaacatccggccggtcgtcgcctccatcaaggagttcttcggcaccagccagctgtcgcagttcatggaccagaacaacccgctgtcgggcctgacgcacaagcgtcgtctcaacgcgctcggcccggggggtctgtcccgtgagcgggccggcttcgaagtccgtgacgtgcacccctcgcact

SEQ ID NO: 124 - DNA, 306 bp, S.violaceoruber; identical to SEQ ID NO: 51

SEQ ID NO: 125 - DNA, 306 bp, S.xanthocidicus
tgcgcaacgtcggcgagctgatccagaaccaggtccgtacgggtctcgcccgtatggagcgcgtcgtgcgtgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcggggctgacgcacaagcgtcgtctgaacgccctcggcccgggtggtctctcccgtgagcgggccggcttcgaggtccgtgacgtgcacccctcgcact

SEQ ID NO: 126 - DNA, 306 bp, S.yokosukanens
tgcgcaacgtcggcgagctgatccagaaccaggtccgtacgggtctcgcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcggggctgacgcacaagcgtcgtctgaacgccctcggcccgggtggtctctcccgtgagcgggccggcttcgaggtccgtgacgtccacccctcgcact

SEQ ID NO: 127 - DNA, 306 bp, S.amakusaensis
tgcgcaacgtcggcgagctgatccagaaccaggtccgtacgggtctggcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcgggtctgacccacaagcgccgtctgtcggcgctgggcccgggtggtctctcccgtgagcgggccggcttcgaggtccgtgacgtgcacccctcgcact

SEQ ID NO: 128 - DNA, 306 bp, S.aburaviensis
tgcgcaacgtcggcgagctcatccagaaccaggtccgtacgggtctggcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcgcagttcatggaccaagaccacccgctgtcgggtctgacccacaagcgccgtctgtccgcgctcggcccgggtggtctctcccgtgagcgcgccggctttgaggtccgtgacgtgcacccctcgcact

SEQ ID NO: 129 - DNA, 306 bp, S.albospinus
tgcgtaacgtcggcgagctcatccagaaccaggtccgtacgggcctggcgcggatggagcgcgtcgtgcgtgagcgcatgaccacgcaggacgtcgaggcgatcacgccgcagaccctgatcaacatccggccggtcgtcgcctccatcaaggagttcttcggcaccagccagctgtcgcagttcatggaccagaacaacccgctgtcgggtctcacccacaagcgccgtctgtcggctcttggcccgggtggtctctcccgtgagcgggccggcttcgaggtccgcgacgtgcacccgtcccact

SEQ ID NO: 130 - DNA, 306 bp, S.albovinaceous
tgcgcaacgtcggcgagctcatccagaaccaggtccgtacgggtctggcgcggatggagcgcgtcgtgcgcgagcgcatgaccacccaggacgtcgaggcgatcacgccgcagaccctgatcaacatccggccggtcgtcgcctccatcaaggagttcttcggcaccagccagctgtcgcagttcatggaccagaacaacccgctgtcgggtctgacccacaagcgccgtctgtcggctcttggcccgggtggtctctcccgtgagcgggccggcttcgaggtccgagacgtgcacccgtcccact

SEQ ID NO: 131 - DNA, 306 bp, S.anabdii
tgcgcagcgtgggcgagctcatccagaaccaggtccgcaccggcctggcccgtatggagcgcgtcgtccgcgagcgcatgacgacccaggacgtcgaggcgatcacgccgcagaccctgatcaacatccggccggtcgtcgcctccatcaaggagttcttcggcaccagccagctgtcccagttcatggaccagaacaacccgctgtccgggctgacgcacaagcggcgtctgtccgcgctcggcccgggtggtctctcccgtgagcgggccggcttcgaggtccgtgacgtgcacccctcgcact

SEQ ID NO: 132 - DNA, 306 bp, S.antibioticus
tccgcaacgtcggcgagctgatccagaaccaggtccgtacgggtctggcccgtatggagcgcgtcgtgcgcgagcgcatgacgacccaggacgtcgaggcgatcacgccgcagaccctgatcaacatccggccggtcgtcgcctccatcaaggagttcttcggcaccagccagctgtcgcagttcatggaccagaacaacccgctgtctggtctgacgcacaagcgccgtctgtcggcgctcggccccggtggtctgtcccgtgagcgggccggcttcgaggtccgtgacgtgcacccctcgcact

SEQ ID NO: 133 - DNA, 306 bp, S.atroolvaceous
tgcgcaacgtcggcgagctcatccagaaccaggtccgtacgggtctggctcgtatggagcgcgtcgtgcgcgagcgcatgacgacccaggacgtcgaggcgatcacgccgcagaccctgatcaacatccggccggtcgtcgcctccatcaaggagttcttcggcaccagccagctgtcccagttcatggaccagaacaacccgctgtcgggtctgacccacaagcgccgtctgtccgcgctgggcccgggtggtctctcccgtgagcgggccggcttcgaggtccgtgacgtgcacccctcgcact

SEQ ID NO: 134 - DNA, 306 bp, S.aureufaciens
tgcgcaacgtcggcgagctcatccagaaccaggtccgtacgggtctggcccgcatggagcgcgtcgtgcgcgagcgcatgaccacccaggacgtcgaggcgatcacgccgcagaccctgatcaacatccggccggtcgtcgcctccatcaaggagttcttcggcaccagccagctgtcgcagttcatggaccagaccaacccgctgtcgggcctgacccacaagcgccgtctgtccgccctcggtccgggtggtctctcccgtgagcgcgccggcttcgaggtccgtgacgtgcacccctcgcact

SEQ ID NO: 135 - DNA, 306 bp, S.azureus
tgcgcaacgtcggcgagctgatccagaaccaggtccgtacgggtctggcgcgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcgcagttcatggaccagaacaacccgctgtcgggtctgacccacaagcgccgtctgtcggcgctgggcccgggaggtctctcccgtgagcgggccggcttcgaggtccgtgacgtgcacccgtcgcact

SEQ ID NO: 136 - DNA, 306 bp, S.baldacii
tgcgcaacgtcggcgagctcatccagaaccaggtccgtacgggtctggcgcgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcgggtctgacccacaagcgccgtctgtcggcgctgggcccgggtggtctctcccgtgagcgggccggcttcgaggtccgtgacgtgcacccctcgcact

SEQ ID NO: 137 - DNA, 306 bp, S.candidus
tgcgcaacgtcggcgagctcatccagaaccaggtccgtacgggtctggcgcgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcgggtctgacccacaagcgccgtctgtcggcgctgggtcccggtggtctgtcccgtgagcgggccggctttgaggtccgagacgtgcacccctcgcact

SEQ ID NO: 138 - DNA, 306 bp, S.caseius
tgcgcaacgtcggcgagctcatccagaaccaggtccgtacgggtctggcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaccaacccgctgtcgggtctgacccacaagcgccgtctgtcggcgctgggcccgggtggtctctcccgtgagcgggccggcttctaggtccgtgacatgcacccgtcccact

SEQ ID NO: 139 - DNA, 306 bp, S.californicus
tgcgcaacgtcggcgagctcatccagaaccaggtccgtacgggtctggcgcgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcgggtctgacccacaagcgccgtctgtcggcgctgggcccgggtggtctctcccgtgagcgggccggcttcgaggtccgtgacgtgcacccgtcgcact

SEQ ID NO: 140 - DNA, 306 bp, S.carpinensis; identical to SEQ ID NO: 40

SEQ ID NO: 141 - DNA, 306 bp, S.chromogenes; identical to SEQ ID NO: 139

SEQ ID NO: 142 - DNA, 306 bp, S.cinnamoneus
tgcgcaacgtcggcgagctcatccagaaccaggtccgtacgggtctggcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcgggtctgacgcacaagcgtcgtctgtccgcgctgggcccgggtggtctgtcccgtgagcgggccggcttcgaggtccgagacgtgcacccgtcgcact

SEQ ID NO: 143 - DNA, 306 bp, S.citreofluorescens; identical to SEQ ID NO: 20

SEQ ID NO: 144 - DNA, 306 bp, S.coerulescens
tgcgcaacgtcggcgagctcatccagaaccaggtccgtacgggtctggcgcgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcgggtctgacgcacaagcgccgtctgtccgcgctcggcccgggtggtctgtcccgtgagcggcccggcttcgaggtccgtgacgtgcacccctcgcact

SEQ ID NO: 145 - DNA, 306 bp, S.coeruleofuscus
tgcgcaacgtcggcgagctgatccagaaccaggtccgtacgggtctggcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcgcagttcatggaccagaacaacccgctgtcgggtctgacccacaagcgccgtctgtcggcgctgggcccgggtggtctgtcccgtgagcgggccggcttcgaggtccgtgacgtgcacccctcgcact

SEQ ID NO: 146 - DNA, 306 bp, S.coralus
tgcgcaacgtcggcgagctcatccagaaccaggtccgtacgggtctggcgcgtatggagcgcgtcgtgcgcgagcgcatgacgacccaggacgtcgaggcgatcacgccgcagaccctgatcaacatccggccggtcgtcgcctccatcaaggagttcttcggcaccagccagctgtcgcagttcatggaccagaacaacccgctgtcgggtctgacccacaagcgtcgtctgtcggcgctgggccccggtggtctgtcccgtgagcgggccggcttcgaggtccgtgacgtgcacccgtcgcact

SEQ ID NO: 147 - DNA, 306 bp, S.cremeus
tgcgcaacgtcggcgagctcatccagaaccaggtccgtacgggtctggcgcgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcgcagttcatggaccagaacaacccgctgtcgggtctgacccacaagcgccgtctgtcggcgctgggccccggtggtctctcccgtgagcgggccggcttcgaggtccgtgacgtgcacccgtcgcact

SEQ ID NO: 148 - DNA, 306 bp, S.cyaneofuscatus
tgcgcaacgtcggcgagctgatccagaaccaggtccgtacgggtctggcgcgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcgcagttcatggaccagaacaacccgctgtcgggtctgacccacaagcgccgtctgtcggcgctgggcccgggtggtctctcccgtgagcgggccggcttcgaggtccgtgacgtgcacccctcgcact

SEQ ID NO: 149 - DNA, 306 bp, S.disatochromogenes
tgcgcagcgtcggcgagctcatccagaaccaggtccgtacgggtctggcgcgtatggagcgtgtcgtccgcgagcgcatgacgacccaggacgtcgaggcgatcacgccccagaccctgatcaacatccggccggtcgtcgcctccatcaaggagttcttcggcaccagccagctgtcgcagttcatggaccagaacaacccgctgtcgggtctgacgcacaagcgtcgtctctcggcactgggtcccggtggtctgtcccgtgagcgggccggcttcgaggtccgtgacgtgcacccgtcccact

SEQ ID NO: 150 - DNA, 306 bp, S.erumpens
tgcgcaacgtcggcgagctcatccagaaccaggtccgtacgggcctggcgcggatggagcgcgtcgtgcgcgagcgcatgaccacccaggacgtcgaggcgatcacgccgcagaccctgatcaacatccggccggtcgtcgcctccatcaaggagttcttcggcaccagccagctgtcgcagttcatggaccagaacaacccgctgtcgggtctcacccacaagcgccgcctgtcggctctcggcccgggtggcctctcccgtgagcgggccggcttcgaggtccgagacgtgcacccgtcccact

SEQ ID NO: 151 - DNA, 306 bp, S.erythraeus
tgcgcaacgtcggcgagctcatccagaaccaggtccgcaccggtctggcccgtatggagcgcgtcgtccgcgagcgcatgaccacgcaggacgtcgaggcgatcacgccgcagaccctgatcaacatccggccggtcgtcgcctccatcaaggagttcttcggcaccagccagctgtcccagttcatggaccagaccaacccgctgtcgggtctgacccacaagcgccgtctgtcggcgctgggcccgggtggtctgagccgtgagcgggcgggcttcgaggtccgtgacgtgcacccgtcccact

SEQ ID NO: 152 - DNA, 306 bp, S.eurythermus
tgcgcaacgtcggcgagctgatccagaaccaggtccgtacgggtctggcccgtatggagcgcgtcgtgcgcgagcgcatgacgacccaggacgtcgaggcgatcacgccgcagaccctgatcaacatccggccggtcgtcgcctccatcaaggagttcttcggcaccagccagctgtcccagttcatggaccagaacaacccgctgtcgggtctgacccacaagcgccgtctgtcggcgctgggccccggtggtctgtcccgtgagcgggccggcttcgaggtccgtgacgtgcacccctcgcact

SEQ ID NO: 153 - DNA, 306 bp, S.fimbriatus
tgcgcagcgtcggcgagctcatccagaaccaggtccgcacgggtctggcgcgtatggagcgcgtcgtgcgcgagcgcatgaccacgcaggacgtcgaggcgatcacgccgcagaccctgatcaacatccggccggtcgtcgcctccatcaaggagttcttcggcaccagccagctgtcccagttcatggaccagaacaacccgctgtccgggctgacgcacaagcgtcgtctgtccgcgctcggcccgggtggtctctcccgtgagcgggccggcttcgaggtccgtgacgtgcacccctcgcact

SEQ ID NO: 154 - DNA, 306 bp, S.flavotricini
tgcgcaacgtcggcgagctcatccagaaccaggtccgtacgggtctggctcgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcgcagttcatggaccagaacaacccgctgtcgggtctgacccacaagcgccgtctgtcggcgctgggcccgggtggtctgtcccgtgagcgggccggcttcgaggtccgtgacgtgcacccgtcgcact

SEQ ID NO: 155 - DNA, 306 bp, S.flavovirens; identical to SEQ ID NO: 127

SEQ ID NO: 156 - DNA, 306 bp, S.fulvissimus
tgcgcaacgtcggcgagctcatccagaaccaggtccgcacgggtctggctcgtatggagcgcgtcgtgcgtgagcgcatgacgacgcaggacgtcgaggcgatcacgccgcagacgctgatcaacatccggccggtcgtcgcctccatcaaggagttcttcggcaccagccagctgtcgcagttcatggaccagaacaacccgctgtcgggtctcacccacaagcgccgcctgtcggctcttggcccgggtggtctctcccgtgagcgggccggcttcgaggtccgtgacgtgcacccgtctcact

SEQ ID NO: 157 - DNA, 306 bp, S.fumanus
tgcgcaacgtcggcgagctgatccagaaccaggtccgtacgggtctggcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcgcagttcatggaccagaacaacccgctgtcgggtctgacccacaagcgccgtctgtcggcgctgggcccgggtggtctctcccgtgagcgggccggcttcgaggtccgtgacgtgcacccctcgcact

SEQ ID NO: 158 - DNA, 306 bp, S.gougeroti
tgcgcaacgtcggcgagctcatccagaaccaggtccgtacgggtctggcgcgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcgggtctgacccacaagcgccgtctgtcggcgctgggccccggtggtctctcccgtgagcgggccggcttcgaggtccgtgacgtgcacccctcgcact

SEQ ID NO: 159 - DNA, 306 bp, S.griseoruber
tgcgcaacgtcggcgagctcatccagaaccaggtccgtacgggtctggcgcgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcgcagttcatggaccagaacaacccgctgtcgggtctgacccacaagcgccgtctgtcggcgctgggcccgggtggtctgtcccgtgagcgggccggcttcgaggtccgtgacgtgcacccgtcgcact

SEQ ID NO: 160 - DNA, 306 bp, S.griseolosporeus
tgcgcaacgtcggcgagctgatccagaaccaggtccgcaccggcctcgcccgcatggagcgggtggtccgcgagcgcatgaccacccaggacgtcgaggcgatcacgccgcaaaccctgatcaacatccggccggtcgtcgcctccatcaaggagttcttcggcaccagccagctgtcgcagttcatggaccagacgaacccgctgtcgggcctgacccacaagcgccgtctgtccgcgctcggcccgggtggtctgtcccgcgagcgggccggcttcgaggtccgtgacgtgcacccctcgcact

SEQ ID NO: 161 - DNA, 306 bp, S.griseostramineus
tgcgcagcgtcggcgagctcatccagaaccaggtccgtacgggtctggcgcgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcgggtctgacccacaagcgccgtctgtcggcgctgggcccgggtggtctctcccgtgagcgggccggcttcgaggtccgtgacgtgcacccctcgcact

SEQ ID NO: 162 - DNA, 306 bp, S.hachijoense; identical to SEQ ID NO: 161

SEQ ID NO: 163 - DNA, 306 bp, S.halstedii; identical to SEQ ID NO: 139

SEQ ID NO: 164 - DNA, 306 bp, S.humidus
tgcgcaacgtcggcgagctgattcagaaccaggtccgtacgggtctggcgcgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcgcagttcatggaccagaacaacccgctgtcgggtctgacccacaagcgccgtctgtcggcgctcggcccgggtggtctctcccgtgagcgggccggcttcgaggtccgtgacgtgcacccctcgcact

SEQ ID NO: 165 - DNA, 306 bp, S.indigoferus
tgcgcaacgtcggcgagctcatccagaaccaggtccgtacgggtctggcccgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcgggtctgacccacaagcgccgtctgtcggcgctgggcccgggtggtctgtcccgtgagcgggccggcttcgaggtccgtgacgtgcacccgtcgcact

SEQ ID NO: 166 - DNA, 306 bp, S.kifunensis
tgcgcagcgtcggcgagctcatccagaaccaggtccgtacgggtctggcgcgtatggagcgcgtcgtgcgcgagcgcatgaccacccaggacgtcgaggcgatcacgccgcagaccctgatcaacatccggccggtcgtcgcctccatcaaggagttcttcggcaccagccagctgtcccagttcatggaccagaacaacccgctgtcgggtctgacccacaagcgccgtctgtcggcgctgggcccgggtggtctctcccgtgagcgggccggctttgaggtccgtgacgtgcacccctcgcact

SEQ ID NO: 167 - DNA, 306 bp, S.kurssanovi
tgcgtaacgtcggcgagctcatccagaaccaggtccgtacgggtctggctcgtatggagcgcgtcgtgcgcgagcgcatgacgacccaggacgtcgaggcgatcacgccgcagaccctgatcaacatccggccggtcgtcgcctccatcaaggagttcttcggcaccagccagctgtcccagttcatggaccagaacaacccgctgtcgggtctgacccacaagcgccgtctgtcggcgctgggcccgggtggtctctcccgtgagcgggccggcttcgaggtccgtgacgtgcacccgtcgcact